(19) United States
(12) Patent Application Publication
(10) Pub. No.: US 2025/0180513 A1
(43) Pub. Date: Jun. 5, 2025

(54) HIGH-THROUGHPUT MULTIPLEXING ELECTROPHORETIC GEL APPARATUS AND RELATED METHODS

(71) Applicants: Cameron O. Zadeh, Greenville, SC (US); Marc R Birtwistle, Central, SC (US); Jonah R Huggins, Central, SC (US)

(72) Inventors: Cameron O. Zadeh, Greenville, SC (US); Marc R Birtwistle, Central, SC (US); Jonah R Huggins, Central, SC (US)

(73) Assignee: Blotting Innovations, LLC, Central, SC (US)

(21) Appl. No.: 18/437,640

(22) Filed: Feb. 9, 2024

Related U.S. Application Data
(63) Continuation-in-part of application No. 17/970,669, filed on Oct. 21, 2022.
(60) Provisional application No. 63/270,436, filed on Oct. 21, 2021.

(51) Int. Cl. G01N 27/447; G01N 1/30; G01N 33/68

(52) U.S. Cl. CPC G01N 27/44782; G01N 1/30; G01N 27/44721; G01N 27/44743; G01N 27/44747; G01N 33/6803

(57) ABSTRACT

A high-throughput multiplexing electrophoretic gel system is disclosed. The system includes a gel casting device which includes an interior, gel casting chamber by which a polymerized gel layer is formed. The polymerized gel layer includes a plurality of integrally formed sample loading wells. The wells are aligned to be simultaneously loaded with samples via an automated microliter multi-pipette sample loader. The sample-loaded gel layer is adapted to undergo immersed horizontal electrophoresis protein separation.

### I. RELATED APPLICATIONS

The present application is a Continuation-in-Part of U.S. Non-provisional patent application Ser. No. 17/970,669, filed on Oct. 21, 2022, which claims priority to U.S. Provisional Patent Application No. 63/270,436, filed on Oct. 21, 2021, the disclosures all of which are hereby incorporated by reference in their entireties.

### II. FIELD OF THE INVENTION

This application discloses claims and embodiments generally related to gel electrophoresis for analyzing biomolecular sample materials, and more particularly, to an improved apparatus, system, and methods for the sequential loading of a plurality of samples for electrophoretic separation of molecules.

### III. BACKGROUND OF THE INVENTION

Western Blotting is a technique and process used in cell and molecular biology to identify proteins from a mixture of proteins extracted from cells. Conventional laboratory equipment utilized to perform western blotting suffers from a number of drawbacks. For example, many known systems require long run times, cannot facilitate high lysate numbers, and/or require very large sample volumes to produce larger sample sizes. Most know systems that produce western blotting higher throughput are complex and consequently expensive, thus rendering them unaffordable for man smaller laboratories, colleges, and research organizations.

Currently there exists in the art, and this industry, a versatile apparatus that is compatible for use with standard western blotting systems which requires only a relatively small sample volume to produce results for a large number of samples.

A search of the prior art did not disclose any patents or publications that read directly on the claims of the instant invention; however, the following references were considered related:
U.S. Pat. No. 11,112,415 B2, issued in the name of Fournier et al.;
U.S. Pat. No. 6,562,213 B1, issued in the name of Cabilly et al.;
U.S. Pat. No. 7,122,104 B2, issued in the name of Cabilly et al.;
U.S. Patent Publication No. 2021/0246440 A1, published in the name of St. Onge et al.;
Zadeh, C. O. et al. Mesowestern Blot: Simultaneous Analysis of Hundreds of Sub-Microliter Lysates. http://biorxiv.org/lookup/doi/10.1101/2021.11.07.467614 (2021) doi:10.1101/2021.11.07.467614; https://pubs.acs.org/doi/full/10.1021/acsomega.2c02201;
Moritz, C. P. 40 years Western blotting: A scientific birthday toast. J. Proteomics 212, 103575 (2020);
Hughes, A. J. et al. Single-cell western blotting. Nat. Methods 11, 749-55 (2014);
Nguyen, U., Squaglia, N., Boge, A. & Fung, P. A. The Simple Western™: a gel-free, blot-free, hands-free Western blotting reinvention. Nat. Methods 8, v-vi (2011);
Ciaccio, M. F., Wagner, J. P., Chuu, C.-B., Lauffenburger, D. A. & Jones, R. B. Systems analysis of EGF receptor signaling dynamics with microwestern arrays. Nat. Methods 7, 148-55 (2010);
Ciaccio, M. F. & Jones, R. B. Microwestern Arrays for Systems-Level Analysis of SH2 Domain-Containing Proteins. Methods Mol Biol 453-473 (2017) doi:10.1007/978-1-4939-6762-9_27;
Towbin, H., Staehelin, T. & Gordon, J. Electrophoretic transfer of proteins from polyacrylamide gels to nitrocellulose sheets: procedure and some applications. Proc. Natl. Acad. Sci. 76, 4350-4354 (1979);
Uhlen, M. et al. A proposal for validation of antibodies. Nat. Methods 13, 823-7 (2016);
Björling, E. & Uhlén, M. Antibodypedia, a portal for sharing antibody and antigen validation data. Mol. Cell. Proteomics MCP 7, 2028-37 (2008);
Bourbeillon, J. et al. Minimum information about a protein affinity reagent (MIAPAR). Nat. Biotechnol. 28, 650-3 (2010);
Tibes, R. et al. Reverse phase protein array: validation of a novel proteomic technology and utility for analysis of primary leukemia specimens and hematopoietic stem cells. Mol. Cancer Ther. 5, 2512-21 (2006);
Hennessy, B. T. et al. A Technical Assessment of the Utility of Reverse Phase Protein Arrays for the Study of the Functional Proteome in Non-microdissected Human Breast Cancers. Clin. Proteomics 6, 129-51 (2010);
Earley, M. C. et al. Report from a workshop on multianalyte microsphere assays. Cytometry 50, 239-42 (2002);
He, J. Practical Guide to ELISA Development. in The Immunoassay Handbook 381-393 (Elsevier, 2013). doi:10.1016/B978-0-08-097037-0.00025-7;
Engvall, E. & Perlmann, P. Enzyme-linked immunosorbent assay, Elisa. 3. Quantitation of specific antibodies by enzyme-labeled anti-immunoglobulin in antigen-coated tubes. J. Immunol. Baltim. Md 1950 109, 129-135 (1972);
Aebersold, R. & Mann, M. Mass-spectrometric exploration of proteome structure and function. Nature 537, 347-55 (2016);
Wilhelm, M. et al. Mass-spectrometry-based draft of the human proteome. Nature 509, 582-7 (2014);
Shevchenko, A., Tomas, H., Havlis, J., Olsen, J. V. & Mann, M. In-gel digestion for mass spectrometric characterization of proteins and proteomes. Nat. Protoc. 1, 2856-60 (2006);
Bittremieux, W. et al. Quality control in mass spectrometry-based proteomics. Mass Spectrom. Rev. 37, 697-711 (2018);
Yates, J. R., Ruse, C. I. & Nakorchevsky, A. Proteomics by Mass Spectrometry: Approaches, Advances, and Applications. Annu. Rev. Biomed. Eng. 11, 49-79 (2009);
Timp, W. & Timp, G. Beyond mass spectrometry, the next step in proteomics. Sci. Adv. 6, eaax8978 (2020);
Handler, D. C. et al. The Art of Validating Quantitative Proteomics Data. PROTEOMICS 18, 1800222 (2018);
Kang, C.-C. et al. Single cell-resolution western blotting. Nat. Protoc. 11, 1508-30 (2016);
Treindl, F. et al. A bead-based western for high-throughput cellular signal transduction analyses. Nat. Commun. 7, 12852 (2016);
Koch, R. J. et al. Validating Antibodies for Quantitative Western Blot Measurements with Microwestern Array. Sci. Rep. 8, 11329 (2018);
Bouhaddou, M. et al. A mechanistic pan-cancer pathway model informed by multi-omics data interprets stochastic cell fate responses to drugs and mitogens. PLoS Comput. Biol. 14, (2018);
Han, Y., Thomas, C. T., Wennersten, S. A., Lau, E. & Lam, M. P. Y. Shotgun Proteomics Sample Processing Automated by an Open-Source Lab Robot. J. Vis. Exp. 63092 (2021) doi:10.3791/63092;
Councill, E. E. A. W. et al. Adapting a Low-Cost and Open-Source Commercial Pipetting Robot for Nanoliter Liquid Handling. SLAS Technol. Transl. Life Sci. Innov. 26, 311-319 (2021);
Villanueva-Cañas, J. L. et al. Implementation of an open-source robotic platform for SARS-CoV-2 testing by real-time RT-PCR. PLOS ONE 16, e0252509 (2021);
Storch, M., Haines, M. C. & Baldwin, G. S. DNA-BOT: a low-cost, automated DNA assembly platform for synthetic biology. Synth. Biol. 5, ysaa010 (2020);
Liu, X., Gygi, S. P. & Paulo, J. A. A Semiautomated Paramagnetic Bead-Based Platform for Isobaric Tag Sample Preparation. J. Am. Soc. Mass Spectrom. 32, 1519-1529 (2021);
Janes, K. A. An analysis of critical factors for quantitative immunoblotting. Sci. Signal. 8, rs2 (2015);
Western Blotting Market|2021-26|Industry Share, Size, Growth—Mordor Intelligence. https://www.mordorintelligence.com/industry-reports/western-blotting-market;
, M. D. F. Western Blotting Market Size, Share, Trends & Growth|2021 to 2026. Market Data Forecast http://www.marketdataforecast.com/;
Western Blotting Market—Global Forecast to 2026|MarketsandMarkets; https://www.marketsandmarkets.com/Market-Reports/western-blotting-market-235810711.html; and
Western Blotting Market Size & Share|Global Industry Report, 2025. https://www.grandviewresearch.com/industry-analysis/western-blotting-market.

This application presents claims and embodiments that fulfill a need or needs not yet satisfied by the products, inventions and methods previously or presently available. In particular, the claims and embodiments disclosed herein describe a high-throughput multiplexing system for conducting electrophoresis separation of molecules, the system comprising a gel casting device and a horizontal electrophoresis tank, the casting device comprises a top portion and a base, the top portion detachably connected to the base and forming an interior, gel casting chamber; the gel casting device further comprises at least one loading port through which a gel solution is introduced into the casting chamber, and wherein the base includes a plurality of protrusions interpolating the gel solution as the gel solution is loaded into the casting chamber, thereby forming a plurality of sample loading wells after polymerization of the gel, the wells each being sequentially auto-loaded with approximately 1.00 μl volume of a liquid sample, the horizontal electrophoresis tank is filled with a buffer solution to a level so as to immerse the sample-loaded polymerized gel, and the tank is configured to enable the sample-loaded polymerized gel to undergo horizontal electrophoresis separation, and wherein the system and device of the present invention providing unanticipated and nonobvious combination of features distinguished from the devices, apparatuses, inventions and methods preexisting in the art. The applicants are unaware of any device, apparatus, method, disclosure or reference that discloses the features of the claims and embodiments disclosed herein, and as more fully described below.

### IV. SUMMARY OF THE INVENTION

In one embodiment, a high-throughput multiplexing electrophoretic gel apparatus is disclosed. The high-throughput multiplexing electrophoretic gel apparatus is adapted and configured to provide a robust, auto or robotic-loadable, precast gel matrix, and is commercially-available and compatible for operational use with standard western blotting systems, and facilitates robust electrophoresis. The apparatus of the present invention comprises a gel casting device comprising a top portion and a bottom portion or base.

According to one embodiment, the top portion and base are separate pieces adapted to be aligned obversely and detachably connected in a closed position. The top portion and base are adapted and configured to be detachably connected in an intimate and complementary, co-planar relationship forming an interior, gel casting chamber.

The gel casting device further comprises at least one loading port through which an unpolymerized, flowable gel solution is introduced into the casting chamber. The upper surface of the base includes a plurality of protrusions which interpolate the gel solution as the gel solution is loaded into the casting chamber. After polymerization of the gel solution, a plurality of sample loading wells is formed integrally within the polymerized gel via the plurality of protrusions.

The vertical gel casting method is utilized to allow the gel solution to polymerize, thus the gel casting device is positioned in a vertical orientation for a standard polymerization period of time, and thereafter producing a polymerized gel layer comprising the plurality of integrally formed sample loading wells.

The plurality of sample loading wells is uniformly spaced and aligned in a geometrical arrangement of linear columns and horizontal rows, thereby enabling samples to be loaded into the plurality of wells via an automated microliter multi-pipette sample loading mechanism, such as the Opentrons® OT-2.

Thereafter, the sample-loaded polymerized gel layer is positioned atop a support base, and the support base and gel layer are placed horizontally in a horizontal electrophoresis tank in order to undergo horizontal electrophoresis separation of molecules. The receptacle of the electrophoresis tank is filled with a buffer solution to a level sufficient to fully immerse the sample-loaded polymerized gel layer. Next, an electric field is applied to the buffer solution so that an electric current passes through the buffer solution, and the sample-loaded polymerized gel layer for a period of approximately thirty to forty-five minutes.

### V. BRIEF DESCRIPTION OF THE DRAWING(S)

The advantages and features of the present invention will become better understood with reference to the following more detailed description and claims taken in conjunction with the accompanying drawings, in which like elements are identified with like symbols, and in which:

FIG. 1 is a front perspective view of a high-throughput multiplexing electrophoretic gel apparatus, in accordance to one embodiment of the present invention;

FIG. 2 is a top plan view of a top portion and base of a gel casting device, in accordance to one embodiment of the present invention;

FIG. 3 is a top plan view of a top portion and base of a gel casting device, in accordance to another embodiment of the present invention;

FIG. 4 is an end view of the gel casting device illustrating two loading ports, in accordance to one embodiment of the present invention;

FIG. 4a is an exploded perspective view of a gel casting device for conducting horizontal gel casting, in accordance to another embodiment of the present invention;

FIG. 4b is a bottom perspective view of the top portion of the gel casting device of FIG. 4a;

FIG. 5 is a top plan view of a gel casting device shown in an opened condition, in accordance to an alternate embodiment of the present invention;

FIG. 6 is an enlarged, partial cross-sectional view of the base showing the plurality of protrusions thereof interpolated through gel solution, in accordance to one embodiment thereof;

FIG. 6a is an enlarged, partial cross-sectional view of a separation medium depicting sample loading wells formed integrally therein, in accordance to one embodiment of the present invention;

FIG. 7 is a top perspective view of a support base supporting a polymerized gel layer disposed with a plurality of sample loading wells, in accordance to one embodiment of the present invention;

FIG. 8 is a perspective view of a conventional laboratory spatula;

FIG. 9 is a front, perspective view of an automated microliter multi-pipette sample loading mechanism;

FIG. 10 is a top perspective view of a horizontal electrophoresis tank, in accordance to one embodiment of the present invention;

FIG. 11 is a top perspective view of a digital scanning and imaging device;

FIG. 12 is a digital image depicting a resultant set of molecular weight ladders, in accordance to one embodiment of the present invention;

FIG. 13 is a digital image showing the resultant separation of U87 cell lysates and intermittent ladder, in accordance to one embodiment of the present invention;

FIG. 14 is a digital image illustrating resultant bands, some of which highlighted with molecular mass measures adjacent thereto, following the transfer of size-separated proteins onto a membrane incubated for beta-actin, and thereafter scanned and imaged, in accordance to one embodiment of the present invention;

FIG. 15 is a digital image showing the metrics utilized for assessing electrophoresis performance, in accordance to one embodiment of the present invention;

FIG. 16 is a digital image illustrating a resultant semi-regular molecular weight ladder following the loading of protein ladder samples into a plurality of sample loading wells in a separation medium, and thereafter being subjected to electrophoresis separation, in accordance to one embodiment of the present invention;

FIG. 17 is an inset detailed view of one set of the separated samples of FIG. 16, wherein the molecular mass measure is provided adjacent each highlighted band, in accordance to one embodiment of the present invention;

FIG. 18 illustrates a commercially-available kit, in accordance to another embodiment of the present invention;

FIG. 19 depicts an alternate embodiment of the present invention;

FIG. 20 is a top perspective, exploded view of a gel casting device, in accordance to another embodiment of the present invention;

FIG. 21 is a top perspective view of the top portion of the gel casting device of FIG. 20;

FIG. 22 is a top perspective view of the base support wall of the gel casting device of FIG. 20 illustrating the perimetric upper surface;

FIG. 23 is an enlarged, partial cross-sectional view of the floor of the top portion of the gel casting device showing the plurality of protrusions thereof interpolated through gel solution, in accordance to one embodiment thereof;

FIG. 23a is an enlarged, partial cross-sectional view of a separation medium depicting sample loading wells formed integrally therein, in accordance to another embodiment of the present invention;

FIG. 24 is a perspective view of a vise;

FIG. 24a is a perspective view of a clamp;

FIG. 25 is a top perspective view of a horizontal electrophoresis tank, in accordance to another embodiment of the present invention;

FIG. 26 is a top perspective view of the horizontal electrophoresis tank in FIG. 25 depicting a polymerized gel layer atop a gel mold support base seated within the support compartment of the tank;

FIG. 27 is a top perspective view of the horizontal electrophoresis tank in FIG. 25 depicting a sample-loaded polymerized gel layer atop a gel mold support base seated within the support compartment of the tank submerged in running buffer;

FIG. 27a is a side elevational view of a collaborative structure, in accordance to one embodiment of the present invention;

FIG. 28 is another side elevational view of the collaborative structure illustrating the separation medium thereof;

FIG. 28a is an enlarged, exploded fragmentary view of a transfer sandwich illustrating the components thereof in sequential stacked alignment, in accordance to one embodiment of the present invention;

FIG. 28b is a perspective view of a blotter tank depicting a transfer sandwich cassette seated therein;

FIG. 28c is a perspective view of a roller of a blotter kit shown in-use;

FIG. 28d is a perspective view of an orbital shaker;

FIG. 28e is an exemplary, commercially-available, digital scanning and imaging device;

FIG. 28f is an enlarged, exploded fragmentary view of a transfer sandwich illustrating the components thereof in sequential stacked alignment, in accordance to another embodiment of the present invention;

FIG. 29 is a digital image illustrating a full membrane scan resultant following the loading of infrared fluorescent molecular weight ladder samples into a plurality of sample loading wells in a separation medium, and thereafter being subjected to electrophoresis separation, in accordance to one embodiment of the present invention;

FIG. 30 is an inset detailed view of one set of the separated samples of FIG. 29, wherein the molecular mass measure is provided adjacent each highlighted band, in accordance to one embodiment of the present invention;

FIG. 31 is a digital image illustrating a full membrane scan resultant following the loading of colorimetric molecular weight ladder samples into a plurality of sample loading wells in a separation medium, and thereafter being subjected to electrophoresis separation, in accordance to one embodiment of the present invention;

FIG. 31a is a digital image illustrating full membrane scans showing replications of the loading of colorimetric molecular weight ladder samples after being loaded into 96 sample loading wells in a 10% polyacrylamide tris-HCl gel layer 74a, and subjected to electrophoresis separation;

FIG. 32 is an inset detailed view of one set of the separated samples of FIG. 31 depicting different molecular weight bands from the ladder, and wherein the molecular mass measure is provided adjacent each highlighted band;

FIG. 32a are digital images illustrating full membrane scans showing replications of the loading of 5 μL, 25 ng of recombinant α-tubulin in a 10% polyacrylamide tris-HCl gel layer 74a, and subjected to electrophoresis separation, and thereafter transferred at 500 mA for 1.5 hours;

FIG. 33 is a digital image illustrating a full membrane scan resultant corresponding to 5 μL of molecular weight ladder samples loaded in each well of the first and last columns of a 10% Tris-HCl gel layer having 96 sample loading wells, and 20 μL/25 ng of recombinant α-Tubulin (˜50 kDa) loaded in the remaining sample loading wells;

FIG. 33a is a digital image illustrating a full membrane scan showing a replication of the loading of 5 μL of molecular weight ladder samples loaded in each well of the first and last columns of a 10% Tris-HCl gel layer having 96 sample loading wells, and 20 μL/25 ng of recombinant α-Tubulin (˜50 kDa) loaded in the remaining sample loading wells;

FIG. 34 illustrates the variation in intensities of the bands in FIG. 33 after being subjected to densitometry;

FIG. 35 illustrates the densitometry results after quantifying the bands in FIG. 33, wherein each band corresponds to a square in the heatmap;

FIG. 36 is a digital image illustrating a full membrane scan resultant corresponding to 5.0 μL of molecular weight ladder samples loaded in each well of the first column and last column (last column not shown) of a 10% Tris-HCl gel layer having 96 sample loading wells, and 4 μL of either recombinant α-Tubulin or HEK293 lysate loaded in the remaining sample loading wells;

FIG. 37 illustrates the variation in intensities of the recombinant α-Tubulin protein bands quantified in FIG. 36 after said bands were subjected to densitometry;

FIG. 38 illustrates the variation in intensities of the HEK293 lysate bands quantified in FIG. 36 after said bands were subjected to densitometry;

FIG. 39 is an enlarged, partial cross-sectional view of a separation medium depicting sample loading wells formed integrally therein, in accordance to another embodiment of the present invention;

FIG. 40 is an enlarged, partial cross-sectional view of a separation medium depicting sample loading wells formed integrally therein, in accordance to another embodiment of the present invention;

FIG. 41 is an enlarged, partial cross-sectional view of a separation medium depicting sample loading wells formed integrally therein, in accordance to still another embodiment of the present invention; and

FIG. 42 is an enlarged, partial cross-sectional view of a separation medium depicting sample loading wells formed integrally therein, in accordance to yet another embodiment of the present invention.

### VI. DETAILED DESCRIPTION OF THE EMBODIMENT(S)

It will be readily understood that the components of the present invention, as generally described and illustrated in the figures herein, may be arranged and designed in a wide variety of different configurations. Thus, the following detailed description of the embodiments, as represented in the attached figures, is not intended to limit the scope of the invention as claimed, but is merely representative of selected embodiments of the invention.

The features, structures, or characteristics of the invention described throughout this specification may be combined in any suitable manner in one or more embodiments. For example, the usage of the phrases “example embodiments”, “some embodiments”, or other similar language, throughout this specification refers to the fact that a particular feature, structure, or characteristic described in connection with the embodiment may be included in at least one embodiment of the present invention. Thus, appearances of the phrases “example embodiments”, “in some embodiments”, “in other embodiments”, or other similar language, throughout this specification do not necessarily all refer to the same group of embodiments, and the described features, structures, or characteristics may be combined in any suitable manner in one or more embodiments.

For purposes of this disclosure, the term “multiplex” or “multiplexing” is intended to be defined as allowing multiple targets quantification in a single sample. For example, multiplex protein assays allow multiple targets quantification in a single sample.

In addition, for purposes of this disclosure, the term “high-throughput”, is defined as an analysis of hundreds or thousands of samples per day in a given laboratory or on a particular instrument.

Further, for purposes of this disclosure, the terms “robust” or “robustness” means the measure of an analytical procedure's capacity to remain unaffected by small, but deliberate variations in method parameters and provides an indication of its reliability during normal usage.

Still further, for purposes of this disclosure, the term “system” means a combination of one or more groups of elements configured to perform one or more functions.

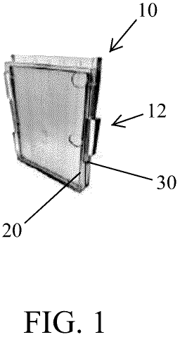

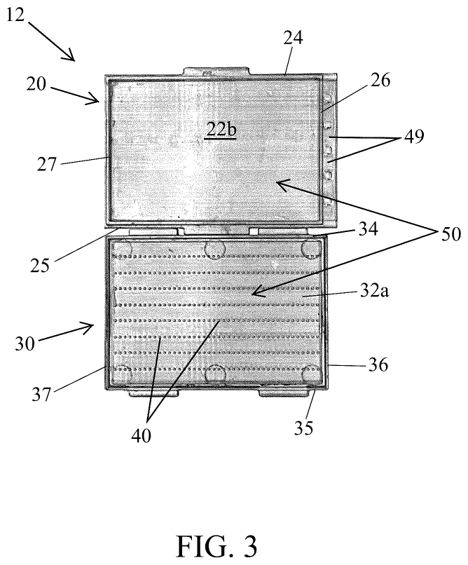

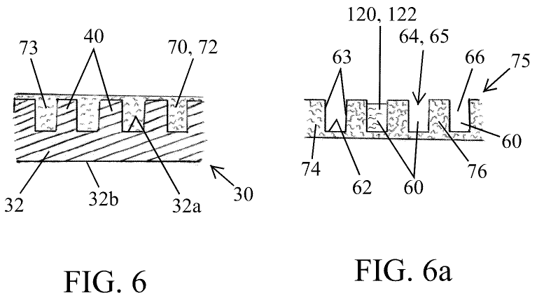

Consistent with the illustrations appended hereto, as embodied in FIGS. 1-7, 10, and 18-19, a high-throughput multiplexing electrophoretic gel apparatus, generally designated at 10 is disclosed, in accordance to one embodiment of the present invention. The high-throughput multiplexing electrophoretic gel apparatus, hereinafter “apparatus 10”, is adapted and configured to provide a robust, manual or robotic-loadable, precast gel matrix, and is commercially-available and compatible for operational use with standard western blotting systems, and facilitates robust electrophoresis.

Referring now more particularly to FIGS. 1-4,6, and 6a, in accordance to one embodiment, the apparatus 10 comprises a gel casting device 12 comprising a generally square or rectangular configuration constructed of a lightweight, rigid material, such as a plastic polymer.

The gel casting device 12 imparts unanticipated and nonobvious functionality to the present invention. More particularly, the gel casting device 12 is adapted and configured to produce an optimum separation medium (gel matrix) for conducting horizontal electrophoresis separation of molecules, particularly sodium dodecyl-sulfate denatured (SDS-denatured) proteins. The separation medium may be integrated with ≥300 sample loading wells (to be described later in greater detail), wherein each loading well is adapted and configured to be loaded with a significantly lower volume of a liquid sample than previously utilized or developed in the art.

In addition, the sample loading wells are uniformly spaced and aligned in a geometrical arrangement of linear columns and horizontal rows within the separation medium, thereby enabling all loading wells to be loaded with samples sequentially via an automated microliter multi-pipette sample loading mechanism.

Further, separation of the molecules of the loaded samples in the wells of the separation medium may be executed by fully immersing the separation medium in a horizontal electrophoresis tank filled with a buffer solution, and thereafter conducting horizontal electrophoresis.

The gel casting device 12 comprises a top portion 20 and a bottom portion or base 30. The top portion 20 comprises a planar top wall 22 having an upper surface 22a opposing a lower surface 22b from which a first longitudinal sidewall 24 opposing a second longitudinal sidewall 25, and a first latitudinal sidewall 26 opposing a second latitudinal sidewall 27 extend integrally downward about a perimeter of the lower surface 22b forming a continuous wall 28. The top wall 22 integrally extends outwardly past first latitudinal sidewall 26 a short distance forming an eave 23, wherein the eave 23 functioning as a handle 23a by which the user may more easily manipulate the gel casting device 12 between open and closed positions.

The base 30 comprises a bottom wall 32 having a lower surface 32b opposing an upper surface 32a from which a first longitudinal sidewall 34 opposing a second longitudinal sidewall 35, and a first latitudinal sidewall 36 opposing a second latitudinal sidewall 37 extend integrally upward about a perimeter of upper surface 32a forming a continuous wall 38.

According to one embodiment, the top portion 20 and base 30 are separate pieces adapted to be aligned obversely and detachably connected in a closed position. The top portion 20 and base 30 are adapted and configured to be detachably connected in an intimate and complementary, co-planar relationship forming an interior, separation medium casting chamber 50. The exterior surface 28a of the continuous wall 28 of the top portion 20 slidably engages the interior surface 38a of the continuous wall 38 of the base 30 in a snug, complementary-fit manner, whereby the top portion 20 is detachably connected to the base 30 via a firm frictional interference fit. In the closed position, the frictionally-engaged continuous walls 28 and 38 of the top portion 20 and base 30, respectively, form a substantially sealed continuous lip edge 54. For purposes of this disclosure, the terms “snug”, “snug-fit”, and “snugly” are each defined as a substantially-intimate, close-fitting relationship.

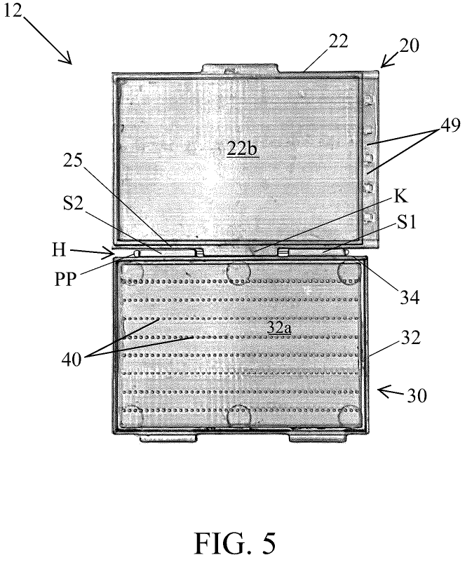

In accordance to another embodiment depicted in FIG. 5, the top portion 20 and base 30 may be hingedly coupled via a hinge mechanism H, wherein the hinge mechanism H may comprise a knuckle K, a pair of shoulders S1 and S2, and a pivot pin PP. The shoulders S1 and S2 project integrally outward, coplanar to the first longitudinal sidewall 34 of the base 30, the shoulders S1 and S2 being aligned spatially parallel. The knuckle K projects integrally outward, coplanar to the second longitudinal sidewall 25 of the top portion 20, and is hingedly coupled between the pair of shoulders S1 and S2. The knuckle K of top portion 20 is hingedly coupled to the pair of shoulders S1 and S2 of the base 30 via the pivot pin PP. The pivot pin PP extends axially through a central hole in the first shoulder S1, through a central hole in the knuckle K, and through a central hole in the second shoulder S2, thereby hingedly coupling top portion 20 to the base 30.

The gel casting device 12 further comprises at least one loading port 49 through which an unpolymerized, flowable separation medium 70 (gel solution) is introduced into the casting chamber 50 of the gel casting device 12 within which the gel solution 70 (i.e., a polyacrylamide gel 72) undergoes vertical gel casting to form a polymerized gel layer 74 in the casting chamber 50. In FIG. 1, the gel casting device 12 is shown positioned in a vertical gel casting orientation.

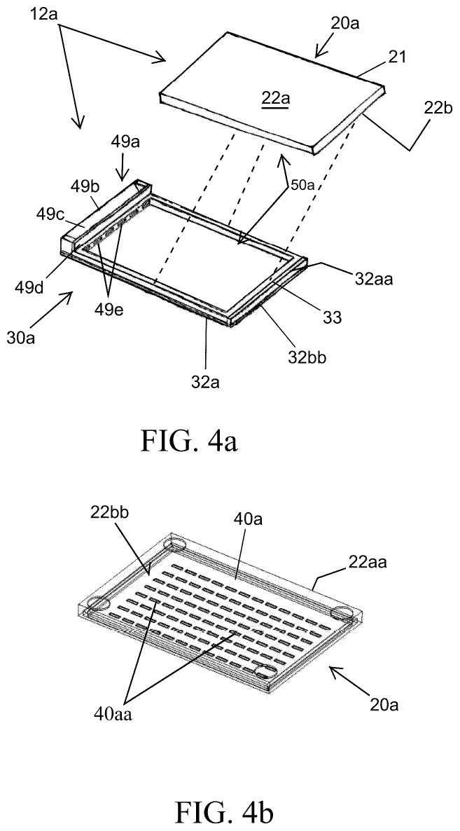

In reference to FIGS. 4a and 4b, and in accordance to another embodiment, a gel casting device 12a for conducting horizontal gel casting is depicted. The gel casting device 12a comprises a generally square or rectangular configuration constructed of a lightweight, rigid material, such as a plastic polymer. The gel casting device 12a comprises a top portion 20a and a bottom portion or base 30a. The base 30a comprises a bottom wall 32a having an upper surface 32aa opposing a lower surface 32bb. The upper surface 32aa of the bottom wall 32a of base 30a comprises a continuous upright wall 33, inset from a perimeter of the upper surface 32aa of the bottom wall 32a. The continuous upright wall 33 encloses a recessed cavity 33a providing a gel molding tray.

The top portion 20a and base 30a are adapted and configured to be detachably connected in an intimate and complementary, co-planar relationship forming an interior, separation medium casting chamber 50a, and which positions the gel casting device 12a in a closed condition.

One end of the bottom portion 30a comprises at least one loading port 49a through which an unpolymerized, flowable separation medium 70 (gel solution) is introduced into the casting chamber 50a of the gel casting device 12 within which the gel solution 70 (i.e., a polyacrylamide gel 72) undergoes vertical gel casting to form a polymerized gel layer 74. In accordance to one embodiment, the at least one loading port 49a comprises an elongated trough 49b having an open top 49c and a sidewall 49d, the sidewall 49d contiguous to continuous upright wall 33. The sidewall 49d of trough 49b comprises a series of outlets 49e through which an unpolymerized, flowable separation medium (gel solution 70) flows into the casting chamber 50a. The trough 49b and series of outlets 49e are in direct, open fluid communication with the casting chamber 50a.

The top portion 20a comprises a top wall 21 comprising an upper surface 22aa opposing a lower surface 22bb. The lower surface 22bb comprises a recessed platform 40a downwardly protruding therefrom. A plurality of protrusions 40aa projects integrally downward from the lower surface 22bb of the platform 40a.

Positioning of top portion 20a and base 30a in a closed condition such that the lower surface 22bb outer periphery edge of the top portion 20a is firmly engaged against the outer periphery edge of the upper surface 32aa of base 30a forms an air-tight sealed lip edge.

Upon loading of the unpolymerized, flowable gel solution 70 through the at least one loading port 49a and into the casting chamber 50a, the plurality of protrusions 40aa interpolate the unpolymerized, flowable gel solution 70. The flowable gel solution 70 comprises a polyacrylamide gel 72, wherein the polyacrylamide gel 72 comprises 10% polyacrylamide tris-glycine gel solution 73, or similar gel solution. Following a standardized polymerization time of sixty minutes, polymerization of the gel solution 70 is effectuated. Significantly, after polymerization of the gel solution 70, a plurality of sample loading wells 60 is formed integrally within the polymerized gel via the plurality of protrusions 40aa. Polymerization of the gel solution 70 produces a polymerized gel layer 74, the polymerized gel layer 74 provides a separation medium 75 for conducting horizontal electrophoresis separation of SDS-denatured proteins.

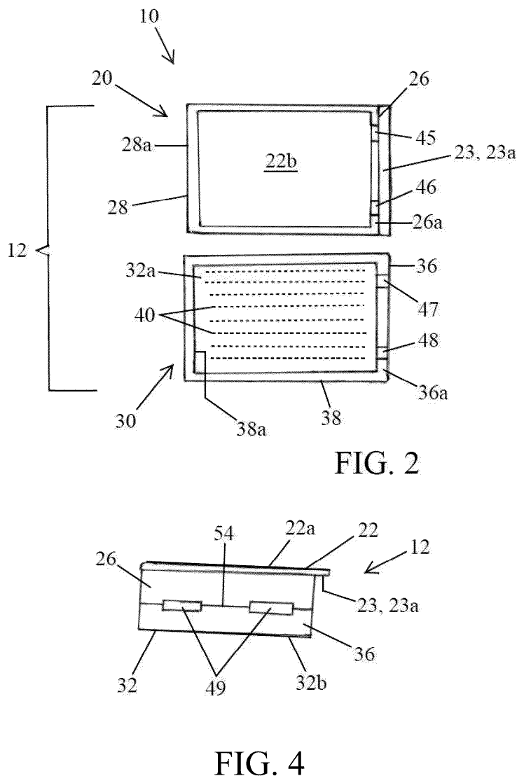

In accordance to one embodiment depicted in FIGS. 2 and 4, a pair of spaced recesses 45 and 46 is disposed along the bottom edge 26a of the first latitudinal sidewall 26 of the top portion 20, and a pair of complementary spaced recesses 47 and 48 is disposed along the upper edge 36a of the first latitudinal sidewall 36 of the base 30. Spaced recesses 45 and 46 and complementary spaced recesses 47 and 48 conjunctively provide a plurality of loading ports 49. More specifically, when the top portion 20 and base 30 are arranged in the closed position (as shown in FIG. 4), spaced recesses 46 and 45 are axially aligned with complementary spaced recesses 47 and 48, respectively, forming a plurality of loading ports 49. The plurality of loading ports 49 are in direct, open fluid communication with the casting chamber 50.

Referring now more particularly to FIGS. 2-3, and 5-6a, a plurality of protrusions 40 integrally projects upwardly from the upper surface 32a of the base 30. Upon loading of the unpolymerized, flowable gel solution 70 through the at least one loading port 49 and into the casting chamber 50, the plurality of protrusions 40 interpolate the unpolymerized, flowable gel solution 70. The flowable gel solution 70 comprises a polyacrylamide gel 72, wherein the polyacrylamide gel 72 comprises 10% polyacrylamide tris-glycine gel solution 73, or similar gel solution. Following a standardized polymerization time of sixty minutes, polymerization of the gel solution 70 is effectuated. Significantly, after polymerization of the gel solution 70, a plurality of sample loading wells 60 is formed integrally within the polymerized gel via the plurality of protrusions 40. Polymerization of the gel solution 70 produces a polymerized gel layer 74, the polymerized gel layer 74 provides a separation medium 75 for conducting electrophoresis separation of SDS-denatured proteins. The polymerized gel layer 74, as previously described, comprises a polyacrylamide gel 72, wherein the polyacrylamide gel 72 comprises 10% polyacrylamide tris-glycine gel matrix 76. The 10% polyacrylamide tris-glycine gel matrix 76 comprises a plurality of sample loading wells 60 formed therein. The polymerized gel layer 74 (gel matrix 76) comprises a thickness measuring in a range comprising approximately 0.50 millimeters (mm) to 2.00 mm, in a range preferably comprising approximately 0.70 mm to 0.90 mm, and most preferably comprising 0.80 mm.

The plurality of sample loading wells 60 is uniformly spaced and aligned in a geometrical arrangement of linear columns and horizontal rows. In accordance to another embodiment, the plurality of sample loading wells 60 may be non-uniformly spaced and aligned. Each of the plurality of sample loading wells 60 comprises a closed bottom 62 opposing an open top 64. A continuous, inner circumferential sidewall 63 extends upward integrally from a periphery of the closed bottom 62 and terminates at the open top 64 forming a generally cylindrical or rectangular space 66 for receiving a volume of a sample 120. The open top 64 provides an inlet aperture 65 through which a sample 120 is loaded.

Each well 60 comprises a sample loading volume measuring in a range comprising approximately 0.50 microliters (μl) to 10.00 μl, in a range preferably comprising approximately 0.75 μl to 5.25 μl, and most preferably comprising approximately 1.00 μl.

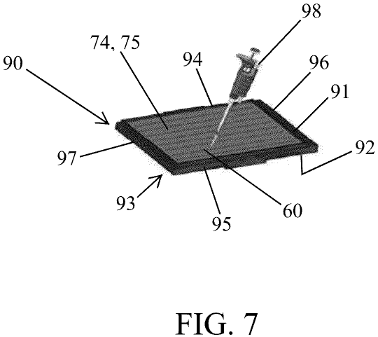

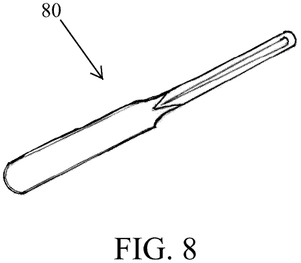

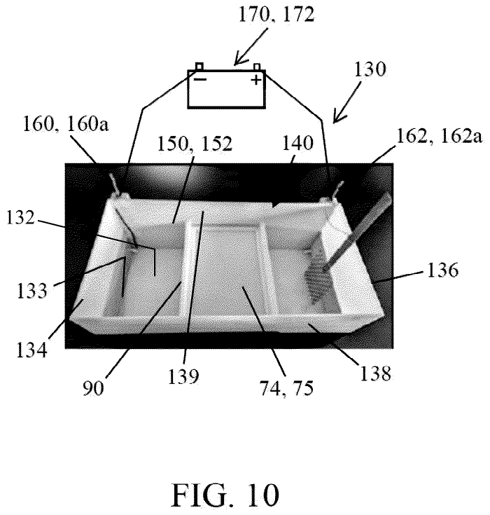

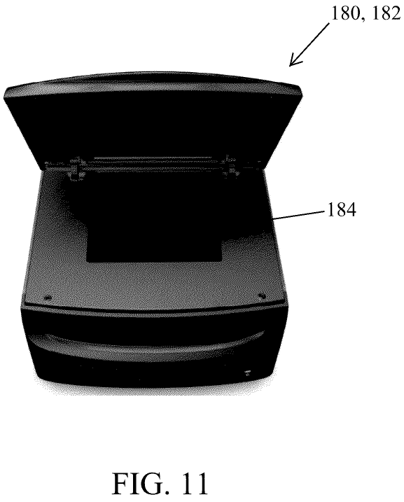

Referring now to FIGS. 7-11, the polymerized gel layer 74 comprises a number of sample loading wells 60, wherein the number of sample loading wells 60 comprises a number in a range comprising approximately 12 to 600, and in a range preferably comprising approximately 48 to 384. Most preferably, the polymerized gel layer 74 comprises at least 96 sample loading wells 60. In reference to FIG. 7, a polymerized gel layer 74 comprising 336 sample loading wells 60 is depicted, wherein each sample loading well 60 therein comprises a sample loading volume measuring approximately 1.00 μl.

The polymerized gel layer 74 is removed from the base 30 using a conventional laboratory spatula 80 and placed superjacent a support base 90, such that the inlet apertures 65 of the sample loading wells 60 of the gel layer 74 are facing upward. The support base 90 comprises a generally square or rectangular configuration constructed of a lightweight, rigid material, such as a plastic polymer. The support base 90 further comprises a planar top wall surface 91 opposing a planar bottom wall surface 92, the planar top wall surface 91 and planar bottom wall surface 92 joined integrally by a plurality of sidewalls 93, wherein the plurality of sidewalls 93 comprises a first longitudinal sidewall 94 opposing a second longitudinal sidewall 95, and a first latitudinal sidewall 96 opposing a second latitudinal sidewall 97. The planar top wall surface 91 defines a surface area dimensionally sized to accommodate the polymerized gel layer 74 within the confines of the periphery of the planar top wall surface 91.

Samples may be manually loaded into the sample loading wells 60 individually using a manual micropipette instrument 98, as shown in FIG. 7. However, hand loading hundreds of sample loading wells 60 manually is tedious, time-intensive, and error-prone.

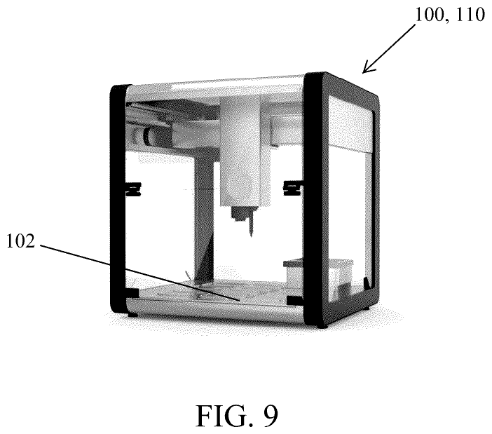

Preferably, samples 120 are loaded via an automated microliter multi-pipette sample loading mechanism 100, such as the Opentrons® OT-2 110 illustrated in FIG. 9. The support base 90 is dimensionally sized, shaped, and configured to sit atop the deck 102 of the automated microliter multi-pipette sample loading mechanism 100 in a size-fit or size-accommodating manner. The uniformly spaced and geometrically aligned arrangement featured by the sample loading wells 60 conforms with spaced intervals between tips on the multi-pipette sample loading mechanism 100, where such loading mechanisms 100 employ a multi-channel pipette, thereby enabling sequential loading of samples 120 in all sample loading wells 60 disposed in the separation medium 75 (10% polyacrylamide tris-glycine gel matrix 76) in a quick, easy, consistent, error-free, and efficient manner.

In accordance to one exemplary embodiment, after seating the support base 90 superjacent the deck 102 of the automated microliter multi-pipette sample loading mechanism 100, liquid samples 120 are loaded into each of the plurality of sample loading wells 60 via the loading mechanism 100. Each liquid sample 120 comprises a volume of 1.00 μl of SDS-denatured protein sample 122. Thus, 1.00 μl of SDS-denatured protein sample 122 is loaded into each sample loading well 60 via the loading mechanism 100.

In order to more easily visualize and quantify the protein bands, a fluorescent protein ladder mixture of proteins pre-stained with fluorescent dye is loaded into each of the sample loading wells 60. The bands fluoresce when the light of a specific wavelength falls on them. Molecular weight markers, or ladders, are a set of standards that are used for determining the approximate size of a protein or a nucleic acid fragment run on an electrophoresis gel. Protein ladders are used to help estimate the size of proteins separated during electrophoresis. They serve as points of reference because they contain mixtures of highly purified proteins with known molecular weights and characteristics. Molecular weight markers or protein ladders also aid in orienting the gel or membrane quickly and monitoring gel migration.

In addition, protein samples prepared for SDS-polyacrylamide gel electrophoresis analysis are denatured by heating in the presence of a sample loading buffer. One exemplary sample buffer is Tris-Glycine sample buffer. Tris-Glycine sample buffer comprises Tris HCl (63 millimoles (mM)), glycerol concentration (10%), SDS (2%), and bromophenol blue concentration (0.0025%), and pH measure of 6.8.

Another exemplary sample buffer is Tris Tricine sample buffer. Tris Tricine sample buffer comprises Tris HCl (450 mM), glycerol concentration (12%), SDS (4%), Coomassie Blue G concentration (0.00075%), phenol red concentration (0.0025%), and pH measure of 8.45.

According to one embodiment, the 1.00 μl SDS-denatured protein sample 122 comprises 2.5 mg/ml of coomassie blue solution ladder. After all sample loading wells 60 have been loaded by the Opentrons® OT-2 110 robots, or manually loaded, the support base 90 with sample-loaded polymerized gel layer 74 positioned thereatop, is placed in a horizontal electrophoresis tank 130. The horizontal electrophoresis tank 130 comprises a bottom wall 132 having a perimetric interface 133 from which a right sidewall 136, a left sidewall 134, a forward sidewall 138, and a rear sidewall 139 extend upward integrally enclosing the bottom wall 132 and forming a receptacle 140 configured to contain a buffer solution 150. The horizontal electrophoresis tank 130 is structurally configured, sizably-shaped, and dimensioned to receive the support base 90 with sample-loaded polymerized gel layer 74 therein in a size-fit manner.

The support base 90 with sample-loaded polymerized gel layer 74 is positioned horizontally atop the bottom wall 132 of the tank 130, in a plane transverse to the bottom wall 132 of the tank 130. The receptacle 140 is then filled with buffer solution 150 to a level sufficient to fully immerse the sample-loaded polymerized gel layer 74 therein. Thereafter, an electric field is applied to the buffer solution 150 so that electric current passes through the buffer solution 150, SDS-denatured protein samples 122, and polymerized gel layer 74.

In order to provide the electric field required to drive electrophoresis separation of molecules, two conductive electrodes 160 and 162, connected electrically to an external power source 170, are coupled to the tank 130 at opposing ends thereof. The two conductive electrodes 160 and 162 comprise a cathode platinum wire 160a and a platinum-coated titanium anode plate 162a, respectively. It is envisioned the two conductive electrodes 160 and 162 may also be constructed of other suitable corrosion and/or degradation resistant metals and alloys, such as stainless steel. The electrical power source 170 comprises a 100 volts (V) direct current (DC) power supply 172. The cathode platinum wire 160a and platinum-coated titanium anode plate 162a is immersed in the buffer solution 150. According to one embodiment, the buffer solution 150 comprises Tris(hydroxymethyl)aminomethane hydrochloride 152.

In order to effect the electrophoresis separation, two opposite ends of the sample-loaded polymerized gel layer 74 (separation medium 75) are exposed to the buffered solution 150. Thereafter, the electrophoretic molecular separation process is triggered by activating the 100V DC power supply 172, thereby applying an electric field to the sample-loaded polymerized gel layer 74. The electrophoretic separation process is conducted approximately 30 to 45 minutes.

After completion of the electrophoretic separation test, the sample-loaded polymerized gel layer 74 is removed from the support base 90 via a conventional laboratory spatula 80 and placed on the interface 184 of a digital scanning and imaging device 180 to detect, identify, and visually produce infrared fluorescence of the molecular weight ladder bands as digital images. In accordance to one embodiment, the digital scanning and imaging device 180 is a LI-COR® Odyssey® Imaging System 182.

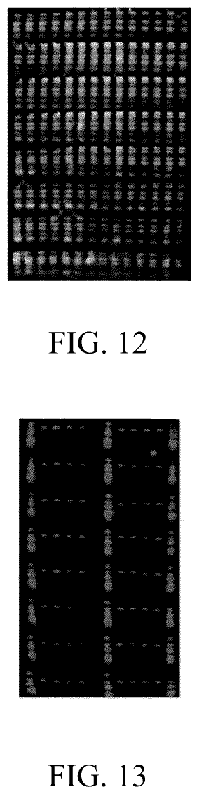

In FIG. 12, the results of the separation of the bands corresponding to the 1.00 μl coomassie blue solution (2.5 mg/ml) ladder samples can be observed. The resultant set of molecular weight ladders illustrated therein was identified following immersed horizontal electrophoresis separation.

In accordance to another embodiment, 1.00 μl of bromophenol blue protein ladder is loaded via the Opentrons® OT-2 device 110 into each of the plurality of sample loading wells 60 of the separation medium 75 (10% polyacrylamide tris-glycine gel matrix 76). The gel matrix 76 is placed in a horizontal electrophoresis tank 130 containing buffer solution 150, wherein the gel matrix 76 being fully immersed in the buffer solution 150. The buffer solution 150 comprises Tris(hydroxymethyl)aminomethane hydrochloride 152. Electrophoresis is triggered by activating the 100V DC power supply 172, thereby applying an electric field to the sample-loaded gel matrix 76. The electrophoretic separation process is conducted approximately 30 to 45 minutes.

In order to normalize the levels of protein detected, a loading control may be utilized and introduced with the samples 120. The loading control operates by confirming that both protein sample loading and protein transfer is equivalent across the polymerized gel matrix 76. The loading control may be selected from the group which includes, but is not limited to, alpha-tubulin and beta-actin.

In accordance to another exemplary embodiment, a number of U87 whole cell lysates and intermittent ladder were loaded via an automated microliter multi-pipette sample loading mechanism 100 into 96 sample loading wells 60 of a polymerized gel matrix 76. The U87 whole cell lysates are provided as a western blotting positive control. The cells are lysed using a Radio-Immunoprecipitation Assay (RIPA) lysis buffer. This process was repeated 16 times, and each sample loaded polymerized gel matrix 76 was subjected to fully immersed horizontal electrophoresis via the horizontal electrophoresis tank 130. Electrophoresis separation was conducted approximately 30 to 45 minutes. Next, the size-separated proteins were transferred onto a membrane, wherein the membrane was blotted for a loading control, namely, alpha-tubulin, and thereafter scanned and imaged via a digital scanning and imaging device 180, wherein the selected digital scanning and imaging device 180 being the LI-COR® Odyssey® Imaging System 182. Alternatively, this step may be described as incubating the membrane with an antibody(ies) recognizing proteins-of-interest and detection antibodies by identifying and isolating each sample loading well 60 by converging on a region of interest, and thereafter scanning and imaging the membrane via a digital scanning and imaging device 180. FIG. 13 is a digital image showing the resultant separation of the U87 cell lysates and intermittent ladder, wherein reproducibility across blocks may be observed.

It is recognized that in the event a running buffer is used to completely submerge the gel matrix 76 when conducting fully immersed horizontal electrophoresis separation, and since the samples 120, 122 are positioned on the top, the inlet aperture 65 of each facing upward, sample wash out may be encountered. To prevent or otherwise eliminate sample wash out, the sample buffer may be adjusted to contain, for example, a higher glycerol percentage to promote higher density and facilitate sinking by the samples 120, 122 into corresponding wells 60.

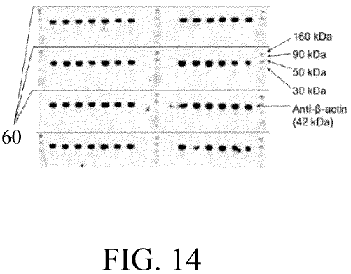

In accordance to another exemplary embodiment, ˜1.00 μl of a lysate was loaded via the automated microliter multi-pipette sample loading mechanism 100 into each of the plurality of sample loading wells 60 of a representative gel matrix 76. The gel matrix 76 was subjected to electrophoresis. The size-separated proteins were transferred onto a membrane, wherein the membrane was incubated for a loading control, namely, beta-actin, and thereafter scanned and imaged via a digital scanning and imaging device 180. FIG. 14 illustrates the resultant bands, some of which highlighted with molecular mass measures adjacent thereto.

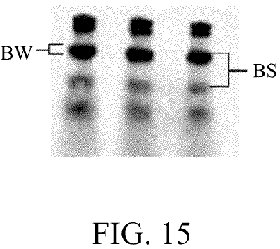

In reference to FIG. 15, the pattern of band separation is a core factor dictating the robustness of electrophoresis. Two key features utilized as metrics for analyzing band separation patterns are band widths and band separation. Band width BW is the measure of the vertical length of each molecular weight band in every lane. Band separation BS is a measure of the vertical length from the midpoint of one band to the midpoint of the next band. FIG. 15 is a digital image showing the band width BW and band separation BS of a molecular weight marker.

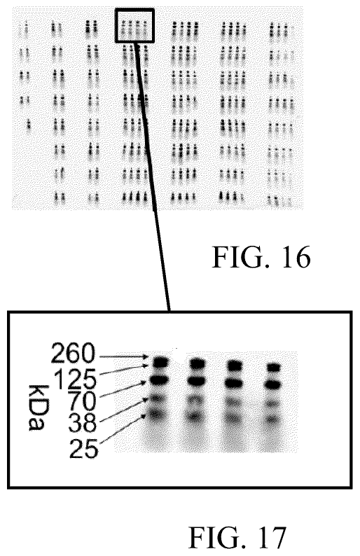

In reference to FIG. 16, a digital image illustrates a resultant semi-regular molecular weight ladder following the loading of protein ladder samples into a plurality of sample loading wells 60 in a separation medium, and thereafter subjected to electrophoresis separation.

FIG. 17 is an inset detailed view of one set of the separated samples of FIG. 16. The molecular mass measure kiloDalton (kDa) (as opposed to molecular weight measure) is provided adjacent each highlighted band.

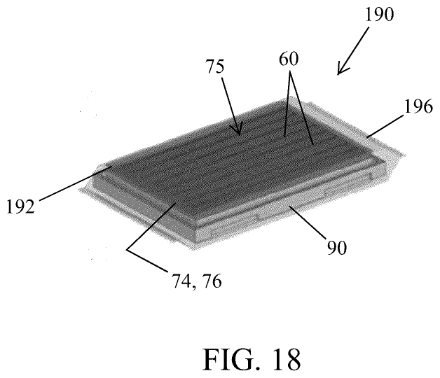

Referring now to FIG. 18, it is envisioned a support base 90, a customized polymerized gel layer 74, and a removably securable cover 192 may be made commercially-available, sold, and shipped as a kit 190. The customized polymerized gel layer 74 comprises a gel being suitable or felicitous to provide a separation medium 75 for conducting electrophoresis separation of molecules, particularly SDS-denatured proteins. In accordance to one embodiment, a suitable gel adapted and configured to provide a separation medium 75 for conducting electrophoresis separation of molecules includes, but is not limited to, 10% polyacrylamide tris-glycine gel 76. The gel 76 includes a plurality of sample loading wells 60, wherein a variety of numbers of wells 60 being available, and also customized in number, thereby being available in a number as selectively-desired and requested by a user/purchaser.

It is envisioned the gel 76 comprises a thickness measuring in a range comprising approximately 0.50 mm to 1.50 mm.

It is further envisioned each well 60 comprises a sample loading volume measuring in a range comprising approximately 0.50 μl to 10.00 μl.

It is still further envisioned the gel 76 comprises a number of sample loading wells 60, wherein the number of sample loading wells 60 comprises a number in a range comprising approximately 10 to 600, and in a range preferably comprising approximately 48 to 384. Most preferably, the polymerized gel 76 comprises approximately 96 sample loading wells 60.

The cover 192 is constructed of a rigid or semi-rigid plastic material and sized so as to effectively cover and shield completely the gel 76 prior to use. Cover 192 is removably attached to the support base 90 via a snap-fit arrangement, frictional-interferential fit, bolts and nuts, screws, clamps, hook-and-loop fasteners, or other suitable attachment mechanism or fastening means.

The support base 90, gel 76, and cover 192 are enclosed or otherwise enveloped entirely and sealed via plastic wrapping 194.

In accordance to still another embodiment of the present invention, a novel system and method for combining a protein-staining membrane with a polyacrylamide gel 72 (as previously described hereinabove regarding a number of exemplary embodiments) to simplify a laboratory technician's process for standard protein transfer from a polyacrylamide gel to a protein-staining membrane. The protein-staining membrane may be selected from the group which includes, but is not limited to, polyvinylidene fluoride (PVDF), Nitrocellulose, or other suitable material. The system and method comprise polyacrylamide gel, a protein-staining membrane, and a thin planar rigid membrane or substrate, such as a plastic card, wherein the card is characterized as a thin and rigid structure. The protein-staining membrane is placed atop the thin plastic card before unpolymerized polyacrylamide gel electrophoresis (SDS-PAGE) gel solution is poured into a gel casting device or apparatus. After being secured in the casting apparatus, the SDS-PAGE gel solution is poured into the casting device and allowed to polymerize to create the final concept.

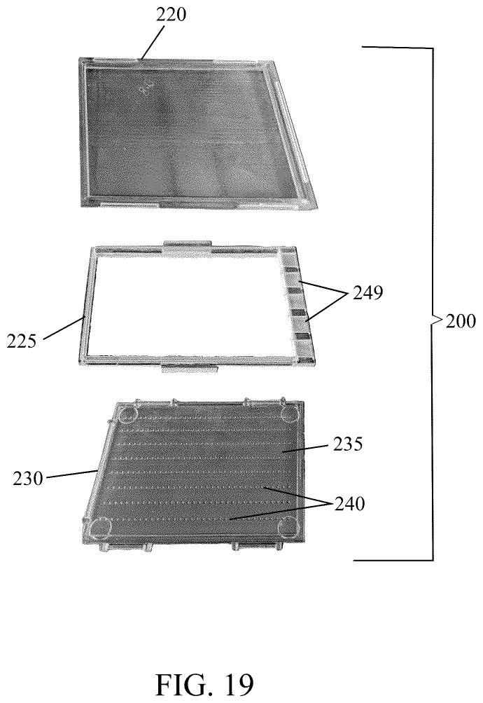

In reference to FIG. 19, an alternate embodiment of the present invention is depicted, wherein a gel casting device 200 comprises a three element structure comprising a top portion 220, a middle portion 225, and a base 230. The top portion 220, middle portion 225, and base 230 are connected in a stacked fashion so as to forms an interior, separation medium casting chamber. Placement of the device 200 in a stacked fashion also places the device 200 in a closed position, thereby forming an interior, separation medium casting chamber.

At least one loading port 249 is integrally formed along one of the latitudinal sidewalls of the middle portion 225. The at least one loading port 249 provides an opening through which an unpolymerized, flowable separation medium (gel solution) is introduced into the casting chamber of the gel casting device 200.

The base 230 comprises a sample wells molding portion 235 for forming a plurality of sample loading wells in the gel solution during polymerization thereof. The molding portion 235 comprises a plurality of protrusions 240 integrally projecting upwardly from the upper surface of the base 230. The top portion 220 and middle portion 225 enclose the gel solution, thereby providing a retaining mold within which polymerized gel is formed.

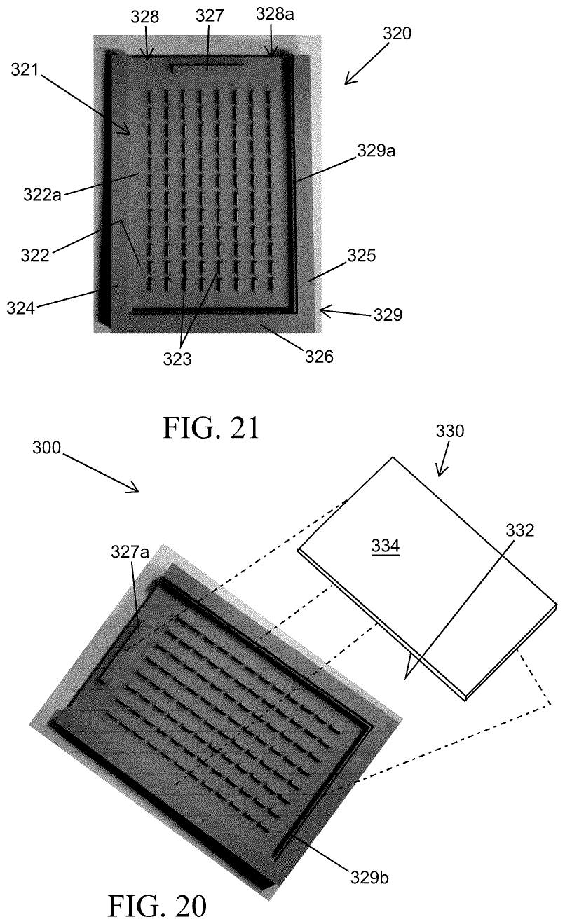

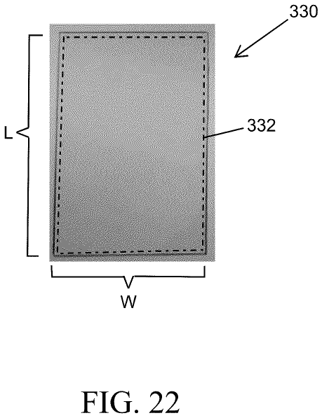

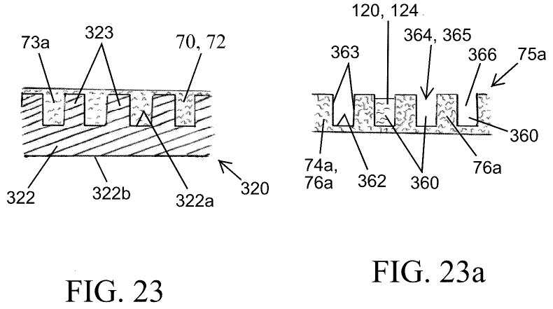

Referring now to FIGS. 20-21, a gel casting device 300 comprises a top portion 320 and a bottom portion or gel mold support base 330. The top portion 320 comprises a sample wells molding portion 321 comprising a floor 322 having an upper surface 322a opposing a lower surface 322b. A first longitudinal sidewall 324 opposing a second longitudinal sidewall 325, and a latitudinal sidewall 326 extend integrally upward about a perimeter of the lower surface 322b of the floor 322 forming an integrated wall 329. An end opposing the latitudinal sidewall 326 is open and includes a base support wall 327 positioned intermediate the first and second longitudinal sidewalls 324 and 325, and adjacent the open-end edge of floor 322, thereby forming a first portal 328 and a second portal 328a through which an unpolymerized, flowable separation medium (gel solution 70) may be introduced (to be described later in greater detail). The top portion 320 further comprises a continuous, support shelf 329a integrally recessed from the first longitudinal sidewall 324, the latitudinal sidewall 326, and the second longitudinal sidewall 325 for seating the base 330 in a complementary-fit manner.

The top portion 320 still further comprises a plurality of protrusions 323 integrally projecting upwardly from the upper surface 322a of the floor 322.

The base 330 comprises a rigid, transparent, planar and generally rectangular configuration. The base 330 is constructed of a thermoplastic polymer, such as including, but not limited to, polycarbonate. The base 330 has a length L measuring approximately 13 centimeters (cm) and a width W measuring approximately 9 cm. The base 330 is positioned superjacent the support shelf 329a and base support wall 327 such that the perimetric upper surface 332 of the base 330 engages the upper edge surfaces 329b, and 327a of the continuous, support shelf 329a, and the base support wall 327, respectively. The gel mold support base 330 is dimensionally sized, shaped, and configured to be seated atop the support shelf 329a in a size-fit or size-compatible manner.

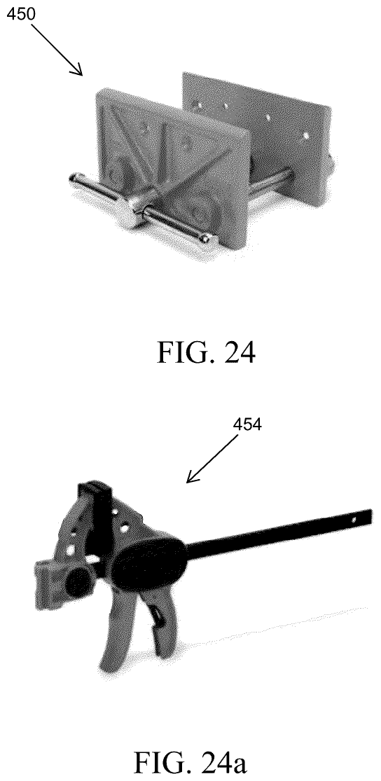

The unpolymerized, flowable gel solution 70 is loaded through the first portal 328 and/or second portal 328a and into the sample wells molding portion 321 or casting chamber 321a. The gel casting device 300 is positioned in a vertical orientation with the first portal 328 and second portal 328a facing upwards (loading edge), and the casting device 300 is held in such vertical orientation via a vise 450, such as the vise illustrated in FIG. 24, sandwiching the end opposing first and second portals 328 and portal 328a, and using slight compressional force. Next, a pair of expandable clamps 454 (one such clamp 454 illustrated in FIG. 24a) were placed and tightened on the loading edge, vertically aligned, and thereafter the vise 450 was fully tightened. Significantly, the order of tightening the vise 450 and clamps 454 helps ensure excess bottom pressure does not force the gel base 330 upwards prior to clamping the loading edge. The plurality of protrusions 323 interpolate the unpolymerized, flowable gel solution 70, and the solution 70 undergoes vertical gel casting to form a polymerized gel layer 74a in the sample wells molding portion 321 or casting chamber.

According to one embodiment, the flowable gel solution 70 comprises a polyacrylamide gel 72, wherein the polyacrylamide gel 72 comprises 10% polyacrylamide Tris-HCl gel solution 73a (250 mL). The flowable unpolymerized 10% Tris-HCl gel solution (250 mL) was prepared using 30% bis/acrylamide (83.33 mL), 1.5 M Tris-HCl, pH 8.8 (62.5 mL), 10% (g/100 mL) SDS (2.5 mL), and to 250 mL with water. 1.5 M Tris-HCl was made by dissolving 181.5 grams (g) of Tris in ˜500 mL of water, then drop dispensing concentrated HCl while monitoring pH with continuous magnetic stirring until pH=8.8, then adding water to 1000 mL. 10% SDS was made by dissolving 10 g of SDS in 80 mL of water with gentle magnetic stirring, then bringing to 100 mL with water. To prepare for gel casting, 70 mL of unpolymerized gel solution, 350 μL of 10% (g/100 mL) ammonium persulfate (APS), and 35 μL of tetramethylethylenediamine (TEMED) were added to a glass beaker and mixed thoroughly. 10% APS was made by dissolving 1 g of APS in 10 mL water and storing at 4° C. Serological pipettes were used to transfer the unpolymerized gel solution 73a with APS and TEMED into the clamped casting assembly 300 slowly. Gels 73a were allowed to polymerize for at least 6 hours but no more than 12 hours. Gels 74a were removed from the casting assembly 300 carefully, a plastic lid placed over the sample loading wells 360 (described in greater detail hereinbelow), and then placed into a vacuum sealing bag with 10 mL of electrophoresis running buffer, and sealed. Gels 74a were stored at 4° C. and used within a month.

As previously described, following a standardized polymerization time of at least 6 hours but no more than 12 hours, polymerization of the gel solution 73a was effectuated, thereby producing a polymerized gel layer 74a, the polymerized gel layer 74a provides a separation medium 75a for conducting electrophoresis separation of proteins, such as including, but not limited to, SDS-denatured proteins and native proteins, and whereby a plurality of sample loading wells 360 is formed integrally within the polymerized gel layer 74a via the plurality of protrusions 323.

Significantly, once the polymerized gel layer 74a has been formed, the gel casting device 300 is placed in an inverted position such that the lower surface 322b of the top portion 320 is oriented obverse and a lower surface 334 of the gel mold support base 330 is laid flat against a horizontal surface (such as a laboratory bench table or countertop). The top portion 320 is removed leaving the base 330 with polymerized gel layer 74a supported thereatop lying on the horizontal surface. The base 330 eliminates the step of transferring the polymerized gel layer 74a from the top portion 320 to the previously-described support base 90 using a laboratory spatula 80 prior to placement of the layer 74a and support base 90 within a horizontal electrophoresis tank 400 (to be described later in greater detail).

In addition, the sample loading wells 360 are uniformly spaced and aligned in a geometrical arrangement of linear columns and horizontal rows within the separation medium 75a, thereby enabling all loading wells 360 to be loaded with samples sequentially via an automated microliter multi-pipette sample loading mechanism 100, such as the Opentrons® OT-2 110 illustrated in FIG. 9.

In accordance to a preferred embodiment of the top portion 320 of the gel casting device 300 depicted in FIGS. 20 and 21, the top portion 320 comprises 8 columns and 12 rows of protrusions 323 totaling 96 protrusions 323, thereby enabling the production of 96 sample loading wells 360. In accordance to other embodiments of the top portion 320 of the gel casting device 300, it is envisioned the top portion 320 is designed and configured to enable the production of a number of sample loading wells 360 which includes, but is not limited to, 12, 24, and 48.

The gel casting device 300 is adapted and configured to produce a polymerized gel layer 74a comprising a length L₁ measuring approximately 13 cm, a width W₁ measuring approximately 8.8 cm, a height H (or thickness) measuring in a range comprising approximately 0.5 millimeters (mm) to 20 mm, and 96 sample loading wells 360, wherein each sample loading well 360 comprises a sample loading volume measuring in a range comprising approximately 1 μl to 1 ml. In accordance to one exemplary embodiment, each sample loading well 360 comprises a sample loading volume measuring 56 μl. In accordance to one exemplary embodiment, the polymerized gel layer 74a comprises a height H (or thickness) measuring approximately 6 mm.

The polymerized gel layer 74a, as previously described, comprises a polyacrylamide gel 72. In accordance to one embodiment, the polyacrylamide gel 72 comprises 10% polyacrylamide tris-HCl gel matrix 76a. The 10% polyacrylamide Tris-HCl gel matrix 76a comprises a plurality of sample loading wells 360 formed therein.

In accordance to another embodiment, the plurality of sample loading wells 360 may be non-uniformly spaced and aligned.

In accordance with one preferred embodiment, each of the plurality of sample loading wells 360 comprises a closed bottom 362 opposing an open top 364. A continuous, inner circumferential sidewall 363 extends upward integrally from a periphery of the closed bottom 362 and terminates at the open top 364 forming a generally rectangular space 366 for receiving a volume of a sample 120. The open top 364 provides an inlet aperture 365 through which a sample 120 is loaded.

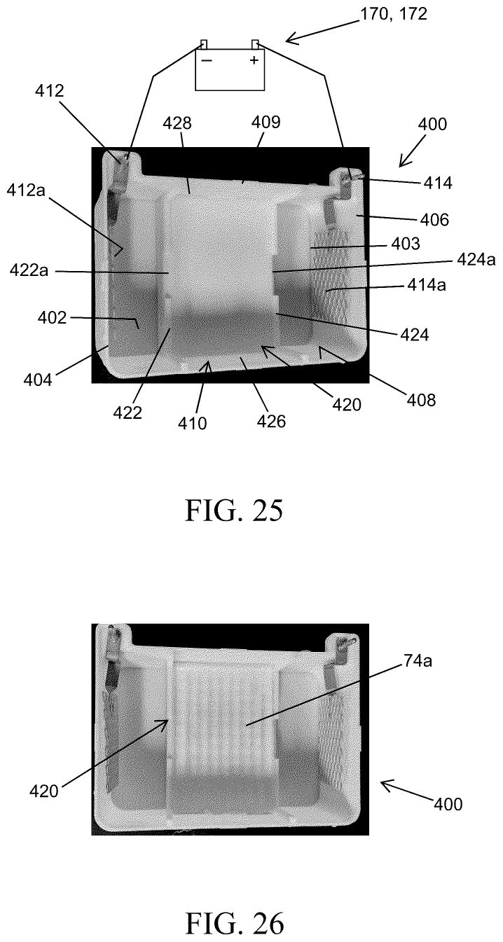

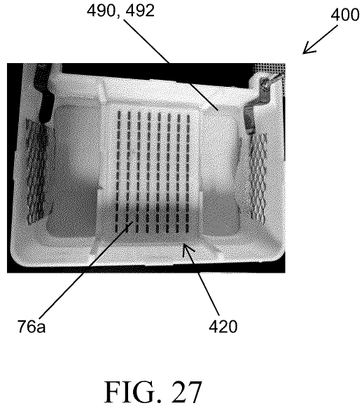

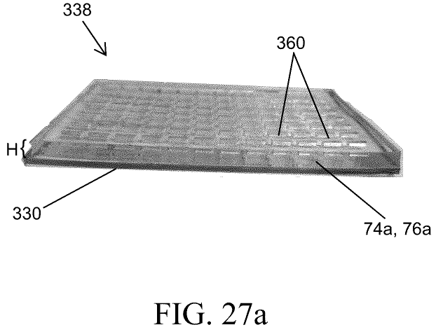

Referring now to FIGS. 25-27, an improved horizontal electrophoresis tank 400 is disclosed, wherein the tank 400 comprises a bottom wall 402 having a perimetric interface 403 from which a right sidewall 406, a left sidewall 404, a forward sidewall 408, and a rear sidewall 409 extend upward integrally enclosing the bottom wall 402 and forming a receptacle 410 configured to contain a buffer solution 430. The horizontal electrophoresis tank 400 further comprises a polymerized gel layer and gel mold support base support compartment 420 positioned intermediate along the bottom wall 402 of the tank 400. The compartment 420 comprises a first longitudinal sidewall 422 opposing a second longitudinal sidewall 424, and a first latitudinal sidewall 426 opposing a second latitudinal sidewall 428. The first longitudinal sidewall 422 and the second longitudinal sidewall 424 each comprises a recessed portion 422a and 424a, respectively, aligned intermediate along the sidewalls 422, 424. The recessed portions 422a and 424a provide spaces enabling the polymerized gel layer 74a and gel mold support base 330 to be grasped by the user in order to be seated in and removed from the polymerized gel layer and gel mold support base support compartment 420 in a quick, easy, and efficient manner. The compartment 420 is structurally configured, sizably-shaped, and dimensioned to receive the gel mold support base 330 and sample-loaded polymerized gel layer 74a in a snug, complementary-fit manner.

The polymerized gel layer 74a, 76a is positioned superjacent the gel mold support base 330, the sample loading wells 360 of the polymerized gel layer 74a are positioned obverse. The polymerized gel layer 74a, 76a and the gel mold support base 330 hereinafter referred to collectively as “collaborative structure 338”. The collaborative structure 338 is seated in the support compartment 420 of the horizontal electrophoresis tank 400 such that the sample loading wells 360 of the polymerized gel layer 74a are positioned obverse. The horizontal electrophoresis tank 400 is filled with buffer solution. In accordance to one embodiment, the buffer solution 490 comprises cold (4° C.) Tris-HCl electrophoresis running buffer (1 Liter (L)) 492 to a depth so as to completely submerge the 10% polyacrylamide tris-HCl gel layer 74a. Next, in order to facilitate loading of liquid samples 120 into each of the sample loading wells 360 of the polymerized gel layer 74a, the samples 120 are manually loaded into the sample loading wells 360 sequentially using a manual micropipette instrument 98, as shown in FIG. 7.

Alternatively, in lieu of manual loading of the samples 120 into the sample loading wells 360, the horizontal electrophoresis tank 400 is positioned superjacent the deck 102 of an automated microliter multi-pipette sample loading mechanism 100, such as the Opentrons® OT-2 110 illustrated in FIG. 9. The horizontal electrophoresis tank 400 is dimensionally sized, shaped, and configured to sit atop the deck 102 of the automated microliter multi-pipette sample loading mechanism 100 in a size-fit or size-accommodating manner. The uniformly spaced and geometrically aligned arrangement featured by the sample loading wells 360 conforms with spaced intervals between tips on the multi-pipette sample loading mechanism 100, where such loading mechanisms 100 employ a multi-channel pipette, thereby enabling sequential loading of samples 120 in all sample loading wells 360 disposed in the separation medium 75a (10% polyacrylamide tris-HCl gel matrix 76a) in a quick, easy, consistent, error-free, and efficient manner.

### Molecular Weight Ladder Experiments

### A. Infrared Fluorescent Molecular Weight Ladder

Liquid samples 120 are manually loaded into the sample loading wells 360 sequentially using a manual micropipette instrument 98.

Alternatively, as previously described in paragraph [00184], liquid samples 120 are loaded into each of the 96 total sample loading wells 360 of a 10% polyacrylamide tris-HCl gel layer 74a via an automated microliter multi-pipette sample loading mechanism 100.

Each liquid sample 120 comprises a volume of 4.5 μL of infrared fluorescent molecular weight ladder 124. The collaborative structure 338 is seated in the support compartment 420 of the horizontal electrophoresis tank 400 such that the sample loading wells 360 of the polymerized gel layer 74a are positioned obverse. The horizontal electrophoresis tank 400 is filled with buffer solution. Electrophoresis was conducted at 50 volts (V) for 45 minutes. Thereafter, the sample-loaded polymerized gel layer 74a was transferred to a nitrocellulose membrane 530, and imaged.

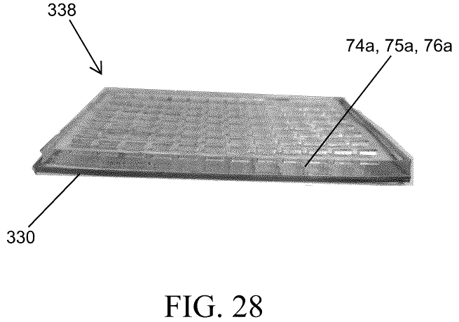

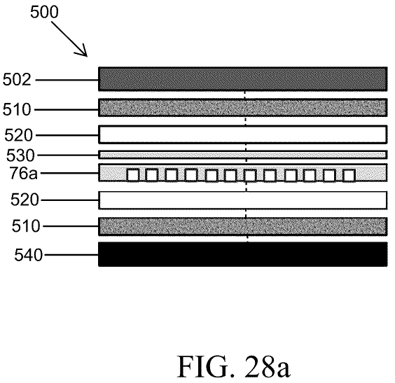

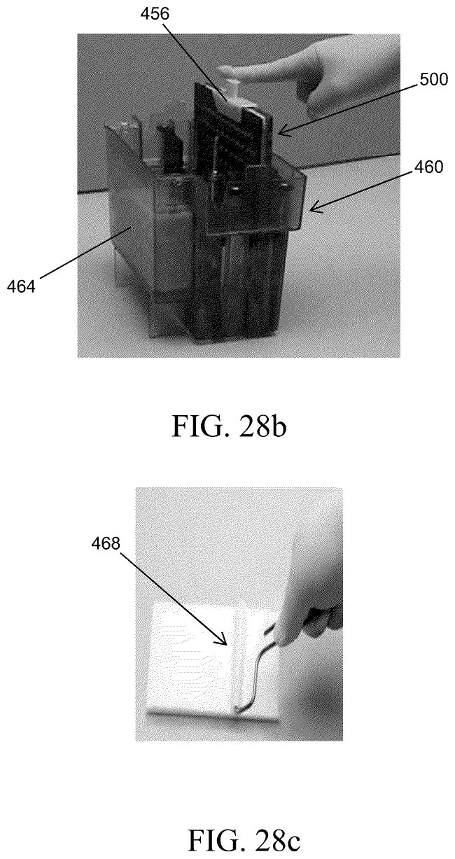

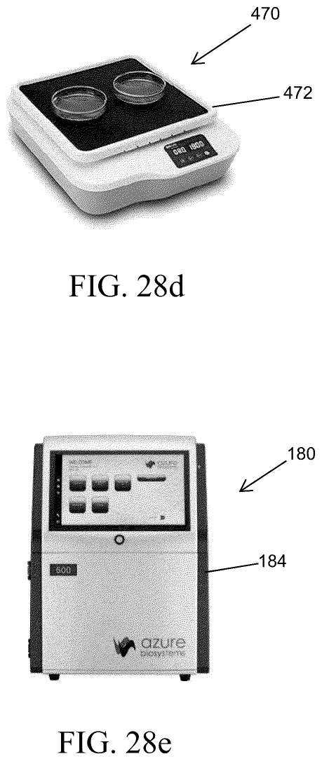

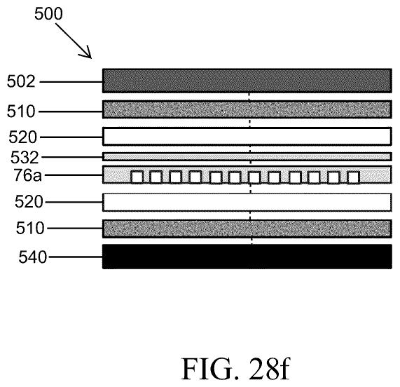

More specifically, after conducting electrophoresis, the collaborative structure 338 was transferred into a plastic tray containing cold transfer buffer and allowed to incubate for 10 minutes. Next, a transfer sandwich 500 (FIG. 28a) was constructed. To construct the transfer sandwich 500, a cold transfer buffer-soaked blotter paper 520 was placed on top of the gel layer 76a (sample loading wells 360 facing upwards), and then placed onto a black transfer cassette edge 540 (black cassette facing down or verso) with a soaked sponge 510 positioned between the cold transfer buffer-soaked blotter paper 520 and the black transfer cassette edge 540, all immersed in cold transfer buffer. Cut-to-size nitrocellulose membrane 530 or PVDF membrane 532 was equilibrated in cold transfer buffer (PVDF 532 was further pre-wetted in methanol), and placed onto the gel layer 76a (flat side thereof without wells 360), and carefully rolled using a roller 468. Cold transfer buffer-soaked blotter paper 520 was placed on top of the nitrocellulose membrane 530, gently rolled via the roller 468, a soaked sponge 510 placed on top of the transfer buffer-soaked blotter paper 520, a red transfer cassette 502 covering the soaked sponge 510, and then the transfer sandwich 500 closed. Care was taken to not pinch or damage the gel layer 76a with transfer sandwich 500 or cassette 502, 540 protrusions or the sliding clamp 456 near edges. The transfer sandwich 500 was placed into a blotter tank 460 in which a magnetic stir bar (not shown) and ice-pack 464 were placed. The blotter tank 460 was filled with cold transfer buffer, magnetic stirring started, and subjected to either constant 500 milliamps (mA) for 90 minutes at room temperature or 30 V for 16 hours at 4° C.

The nitrocellulose membrane 530 was removed from the sandwich 500 and inspected for efficient molecular weight ladder transfer. If only molecular weight ladder was loaded, the membrane 530 was then imaged (to be described in greater detail below). In the event other samples were present, the membrane 530 was immediately placed into a glass dish containing 20 mL of room temperature blocking buffer, and subjected to 100 rpm on an orbital shaker 470 for 1 hour. In accordance to one embodiment, the orbital shaker 470 is an ONiLAB® orbital shaker 472 (see FIG. 28d). Blocking buffer was prepared by adding 5 g non-fat dry milk to 100 mL of TBS-T (Tris Buffered Saline with Tween-20, prepared as follows). A 10×TBS stock (1 L) was prepared by dissolving 24 g Tris and 88 g NaCl in ˜500 mL of water, adjusting pH to 7.6 as previously described with HCl, and then adding water to 1 L. A 10% (v/v) Tween-20 solution was prepared by adding 10 mL Tween-20 to 90 mL water. A 1×TBS-T solution (1 L) was prepared by mixing 100 mL 10×TBS into 890 mL water, then adding 10 mL of 10% Tween-20.

After blocking, the membrane 530 was incubated with 20 mL of primary antibody solution at room temperature for 1 hour at 100 rpm. The primary antibody solution was prepared by adding anti-α-Tubulin 1:5,000 (v/v) to blocking buffer. The membrane 530 was then washed 3 times with ˜20 mL of TBS-T for 5 minutes at 100 rpm at room temperature. Following washing, the membrane 530 was incubated with 20 mL of secondary antibody solution at room temperature for 1 hour at 100 rpm. The membrane 530 was then washed 3 times with ˜20 mL of TBS-T for 5 minutes at 100 rpm at room temperature. After the final wash, ˜2 mL of chemiluminescent substrate solution was prepared according to manufacturer's instructions, and was applied dropwise to the top of the membrane 530 to prepare for imaging.

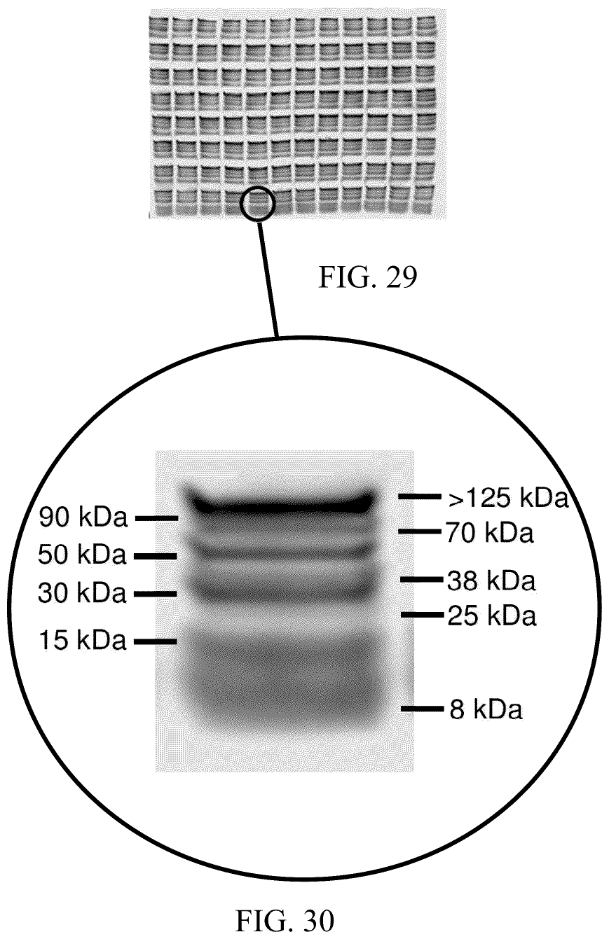

The nitrocellulose membrane 530 is placed on the interface 184 (scanner bed) of a digital scanning and imaging device 180 to detect, identify, and visually produce infrared fluorescence of the molecular weight ladder bands as digital images. Both 700 nm and 800 nm channels were acquired with 169 μm resolution. The resulting image was converted to grayscale for presentation in FIGS. 29 and 30. In accordance to one embodiment, the digital scanning and imaging device 180 is a LI-COR® Odyssey® Imaging System 182. FIG. 29 is a digital image illustrating a full membrane scan resultant corresponding to the 4.5 μL of infrared fluorescent molecular weight ladder samples. FIG. 30 is an inset detailed view of one set of the separated samples of FIG. 29. Different molecular weight bands from the ladder are indicated, wherein the molecular mass measure is provided adjacent each highlighted band.

The Tris-HCl running buffer (1 L) was prepared by dissolving 12.1 g of Tris in ˜500 mL water, drop dispensing HCl to pH 7.5, adding water to 990 mL, and then 10 mL 10% SDS.

Prior to conducting the experiments, the transfer buffer was prepared by (i) adding 30.3 g Tris and 144 g glycine into a 2 L glass beaker, (ii) adding ˜500 mL double distilled water (ddH₂O) into the 2 L glass beaker, (iii) adding a magnetic stir bar into the 2 L glass beaker, (iv) placing the 2 L glass beaker on a stir plate, (v) setting the stir plate to a gentle agitation mode and allowing the stir plate to gently agitate over a period of time sufficient to dissolve the 30.3 g Tris and 144 g glycine. It is noted that several hours may be required to dissolve the 30.3 g Tris and 144 g glycine into a solution. Once the 30.3 g Tris and 144 g glycine have been dissolved into a solution, said solution is transferred to a 1 L graduated cylinder into which ddH₂O is added to 800 mL, thereby producing a 10× stock solution which is stored at room temperature.

In order to provide and ensure an ample supply of cold buffer, the day before transfer of the collaborative structure 338, 160 mL of 10× stock solution is added into a 2 L graduated cylinder. 400 mL methanol is then added into the 2 L graduated cylinder, and ddH₂O is added to 2000 mL, thereby producing a 1× stock solution which is stored at 4° C.

After electrophoresis, the gel layer 76a with gel mold support base 330 (collaborative structure 338) was transferred into a plastic tray containing cold transfer buffer and allowed to incubate for 10 minutes.

The infrared fluorescent molecular weight ladder 124 samples were prepared by mixing 500 μL of infrared fluorescent molecular weight ladder with 20 μL of 0.1% (g/100 mL) bromophenol blue loading dye solution and 30 μL of 100% glycerol and 4.5 μL loaded per well 360. Bromophenol blue loading dye solution (10 mL) was prepared with 0.01 g bromophenol blue, 5 mL of 50% glycerol, 0.5 mL of 10% sodium dodecyl sulfate (SDS), 2.1 mL of 1.5M Tris-HCl, and water to 10 mL.

### B. Colorimetric Molecular Weight Ladder

The horizontal electrophoresis tank 400 is filled with buffer solution 490 (cold (4° C.) Tris-HCl electrophoresis running buffer (1 Liter (L)) 492 to a depth so as to completely submerge the 10% polyacrylamide tris-HCl gel layer 74a. Liquid samples 120 are manually loaded into the sample loading wells 360 sequentially using a manual micropipette instrument 98.

Alternatively, as previously described in paragraph [00184], liquid samples 120 are loaded into each of the 96 total sample loading wells 360 of a 10% polyacrylamide tris-HCl gel layer 74a via an automated microliter multi-pipette sample loading mechanism 100.

Each liquid sample 120 comprises a volume of 5.0 μL of colorimetric molecular weight ladder 126. Electrophoresis was conducted at 50 volts (V) for 45 minutes. Thereafter, the sample-loaded polymerized gel layer 74a was transferred to a PVDF membrane 532, and imaged.

More particularly, after conducting electrophoresis, the collaborative structure 338 was transferred into a plastic tray containing cold transfer buffer and allowed to incubate for 10 minutes. Next, a transfer sandwich 500 was constructed. To construct the transfer sandwich 500, a cold transfer buffer-soaked blotter paper 520 was placed on top of the gel layer 76a (sample loading wells 360 facing upwards), and then placed onto a black transfer cassette edge 540 (black cassette facing down or verso) with a soaked sponge 510 positioned between the cold transfer buffer-soaked blotter paper 520 and the black transfer cassette edge 540, all immersed in cold transfer buffer. Cut-to-size PVDF membrane 532 was equilibrated in cold transfer buffer and further pre-wetted in methanol), and placed onto the gel layer 76a (flat side thereof without wells 360), and carefully rolled using the roller 468. Cold transfer buffer-soaked blotter paper 520 was placed on top of the PVDF 532, gently rolled via the roller 468, a soaked sponge 510 placed on top of the transfer buffer-soaked blotter paper 520, a red transfer cassette 502 covering the soaked sponge 510, and then the transfer sandwich 500 closed. Next, the transfer sandwich 500 was placed into a blotter tank 460 in which a magnetic stir bar (not shown) and ice-pack 464 were placed. The blotter tank 460 was filled with cold transfer buffer, magnetic stirring started, and subjected to either constant 500 milliamps (mA) for 90 minutes at room temperature or 30 V for 16 hours at 4° C.

The PVDF membrane 532 was removed from the sandwich 500 and inspected for efficient molecular weight ladder transfer. If only molecular weight ladder was loaded, the membrane 532 was then imaged. In the event other samples were present, the membrane 532 was immediately placed into a glass dish containing 20 mL of room temperature blocking buffer, and subjected to 100 rpm on an orbital shaker 470 for 1 hour. In accordance to one embodiment, the orbital shaker 470 is an ONiLAB® orbital shaker 472 (see FIG. 28d).

After blocking, the membrane 532 was incubated with 20 mL of primary antibody solution at room temperature for 1 hour at 100 rpm. The primary antibody solution was prepared by adding anti-α-Tubulin 1:5,000 (v/v) to blocking buffer. The membrane 532 was then washed 3 times with ˜20 mL of TBS-T for 5 minutes at 100 rpm at room temperature. Following washing, the membrane 532 was incubated with 20 mL of secondary antibody solution at room temperature for 1 hour at 100 rpm. The membrane 532 was then washed 3 times with ˜20 mL of TBS-T for 5 minutes at 100 rpm at room temperature. After the final wash, ˜2 mL of chemiluminescent substrate solution was prepared according to manufacturer's instructions, and was applied dropwise to the top of the membrane 532 to prepare for imaging.

The PVDF membrane 532 is placed onto a black chemi tray which was placed onto the top shelf in the digital scanning and imaging device 180 to detect, identify, and visually produce chemiluminescence of the molecular weight ladder bands as digital images. In accordance to one embodiment, the digital scanning and imaging device 180 is an Azure 300 Avantor® 184 (see FIG. 28e).

Acquisition was done via the Chemi Blot module with automatic exposure time calculation, cumulative image generation, and color marker selected. Any images with saturated pixels were discarded. Images were saved as .jpg for figure generation and .tiff for quantitative analysis.

For quantitative analysis of chemiluminescent bands, a Java-based image processing program, namely ImageJ, was used. A rectangle region of interest was placed over one row of bands at a time. Under “Analyze Gels”, “select first lane” was chosen, and then plot lanes. On the generated intensity profile plots, vertical lines were drawn manually to separate peaks, and then the wand tool was used by clicking inside each peak to generate the area metric for each band. This process was repeated for each row in a blot image, and arbitrary area units were scaled to be unitless as described in specific results.

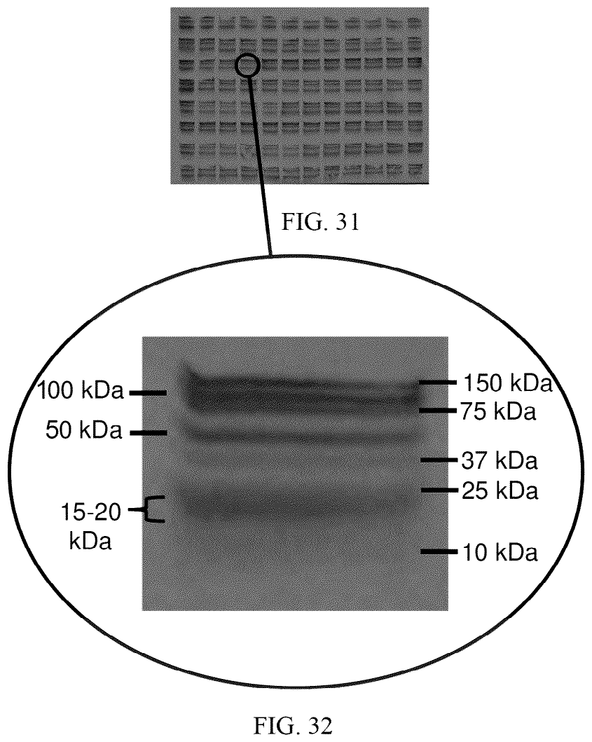

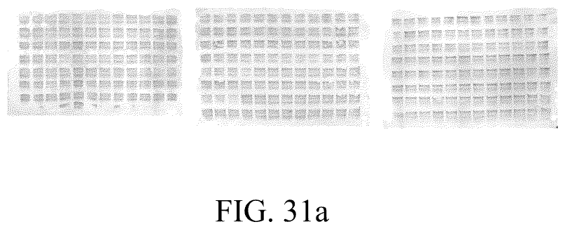

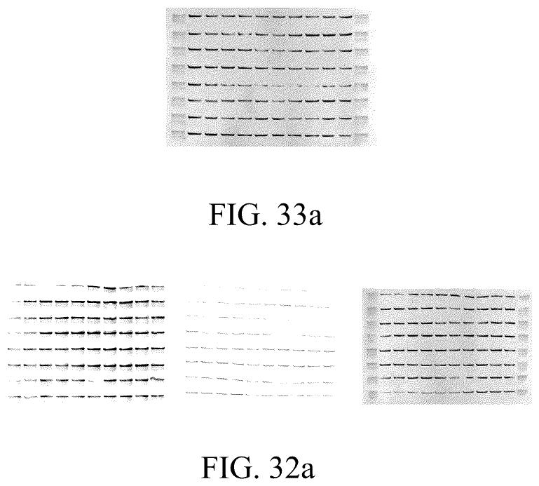

FIG. 31 is a digital image illustrating a full membrane scan resultant corresponding to the 5.0 μL of colorimetric molecular weight ladder samples. FIG. 32 is an inset detailed view of one set of the separated samples of FIG. 31. Different molecular weight bands from the ladder are indicated, wherein the molecular mass measure is provided adjacent each highlighted band.

FIG. 31a is a digital image illustrating full membrane scans showing replications of the loading of colorimetric molecular weight ladder samples after being loaded into 96 sample loading wells 360 in a 10% polyacrylamide tris-HCl gel layer 74a, and subjected to electrophoresis separation.

FIG. 32a are digital images illustrating full membrane scans showing replications of the loading of 5 μL, 25 ng of recombinant α-tubulin in a 10% polyacrylamide tris-HCl gel layer 74a, and subjected to electrophoresis separation, and thereafter transferred at 500 mA for 1.5 hours.

The colorimetric molecular weight ladder was mixed 1:1 (v/v) with clear sample buffer and 5 μL loaded per well. Clear sample buffer (1 mL) was prepared with 500 μL of 50% (v/v water) glycerol, 200 μL 10% SDS, 42 μL 1.5 M Tris-HCl, 50 μL 2-Mercaptoethanol, and water to 1 mL.

### C. Recombinant α-Tubulin or HEK293 Cell Lysate

The horizontal electrophoresis tank 400 is filled with buffer solution 490 (cold (4° C.) Tris-HCl electrophoresis running buffer (1 Liter (L)) 492 to a depth so as to completely submerge the 10% polyacrylamide tris-HCl gel layer 74a. Liquid samples 120 are manually loaded into each of the 96 total sample loading wells 360 sequentially using a manual micropipette instrument 98.

Alternatively, as previously described in paragraphs [00184] and [00196], liquid samples 120 are loaded into the sample loading wells 360 of a 10% polyacrylamide tris-HCl gel layer 74a via an automated microliter multi-pipette sample loading mechanism 100. More specifically, a liquid sample 120 comprising a volume in a range measuring approximately of 0.1 to 1 ml of molecular weight ladder 127 was loaded in each well 360 of the first and last columns of the 10% polyacrylamide tris-HCl gel layer 74a, and lysate or other selectively-desired protein-containing liquid sample 120 was loaded in the wells 360 between the first column and the last column of the tris-HCl gel layer 74a. In accordance to one exemplary embodiment, 5 μL of molecular weight ladder 127 was loaded in each well 360 of the first and last columns of the 10% polyacrylamide tris-HCl gel layer 74a, and 20 μL/25 ng of recombinant α-Tubulin (˜50 kDa) 128 in the other wells 360 of the tris-HCl gel layer 74a.

Electrophoresis was conducted, and thereafter, the sample-loaded polymerized gel layer 74a was transferred to a PVDF membrane 532, and imaged.

More particularly, after conducting electrophoresis, the collaborative structure 338 was transferred into a plastic tray containing cold transfer buffer and allowed to incubate for 10 minutes. Next, a transfer sandwich 500 was constructed. The cold transfer buffer-soaked blotter paper 520 was placed on top of the gel layer 76a (sample loading wells 360 facing upwards), and then placed onto a black transfer cassette edge 540 (black cassette facing down or verso) with a soaked sponge 510 positioned between the cold transfer buffer-soaked blotter paper 520 and the black transfer cassette edge 540, all immersed in cold transfer buffer. PVDF membrane 532 was equilibrated in cold transfer buffer and further pre-wetted in methanol), and placed onto the gel layer 76a (flat side thereof without wells 360), and carefully rolled using the roller 468. Cold transfer buffer-soaked blotter paper 520 was placed on top of the PVDF 532, gently rolled via the roller 468, a soaked sponge 510 placed on top of the transfer buffer-soaked blotter paper 520, a red transfer cassette 502 covering the soaked sponge 510, and then the transfer sandwich 500 closed. Next, the transfer sandwich 500 was placed into a blotter tank 460 in which a magnetic stir bar (not shown) and ice-pack 464 were placed. The blotter tank 460 was filled with cold transfer buffer, magnetic stirring started, and subjected to constant 30 V for 16 hours at 4° C.

The PVDF membrane 532 was removed from the sandwich 500 and inspected for efficient molecular weight ladder transfer. In view of both molecular weight ladder 127 and recombinant α-Tubulin 128 being loaded, the membrane 532 was immediately placed into a glass dish containing 20 mL of room temperature blocking buffer, and subjected to 100 rpm on an orbital shaker 470, 472 for 1 hour.

After blocking, the membrane 532 was incubated with 20 mL of primary antibody solution at room temperature for 1 hour at 100 rpm. The membrane 532 was then washed 3 times with ˜20 mL of TBS-T for 5 minutes at 100 rpm at room temperature. Following washing, the membrane 532 was incubated with 20 mL of secondary antibody solution at room temperature for 1 hour at 100 rpm. The membrane 532 was then washed 3 times with ˜20 mL of TBS-T for 5 minutes at 100 rpm at room temperature. After the final wash, ˜2 mL of chemiluminescent substrate solution was prepared according to manufacturer's instructions, and was applied dropwise to the top of the membrane 532 to prepare for imaging.

The PVDF membrane 532 is placed onto a black chemi tray which was placed onto the top shelf in the digital scanning and imaging device 180 to detect, identify, and visually produce chemiluminescence of the molecular weight ladder bands as digital images. In accordance to one embodiment, the digital scanning and imaging device 180 is an Azure 300 Avantor® 184 (see FIG. 28e).

Acquisition was done via the Chemi Blot module with automatic exposure time calculation, cumulative image generation, and color marker selected. Any images with saturated pixels were discarded. Images were saved as .jpg for figure generation and .tiff for quantitative analysis.

For quantitative analysis of chemiluminescent bands, a Java-based image processing program, namely ImageJ, was used. A rectangle region of interest was placed over one row of bands at a time. Under “Analyze Gels”, “select first lane” was chosen, and then “plot lanes”. On the generated intensity profile plots, vertical lines were drawn manually to separate peaks, and then the wand tool was used by clicking inside each peak to generate the area metric for each band. This process was repeated for each row in a blot image, and arbitrary area units were scaled to be unitless as described in specific results.

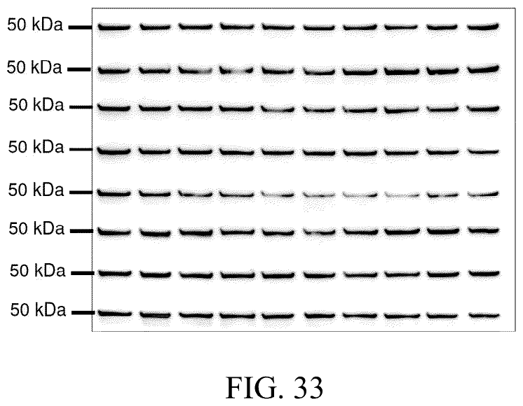

FIG. 33 is a digital image illustrating a full membrane scan resultant corresponding to 5 μL of molecular weight ladder samples loaded in each well 360 of the first and last columns of a 10% Tris-HCl gel layer having 96 sample loading wells 360, and 20 μL/25 ng of recombinant α-Tubulin (˜50 kDa) loaded in the remaining sample loading wells 360.

FIG. 33a is a digital image illustrating a full membrane scan showing a replication of the loading of 5 μL of molecular weight ladder samples loaded in each well 360 of the first and last columns of a 10% Tris-HCl gel layer having 96 sample loading wells 360, and 20 μL/25 ng of recombinant α-Tubulin (˜50 kDa) loaded in the remaining sample loading wells 360.

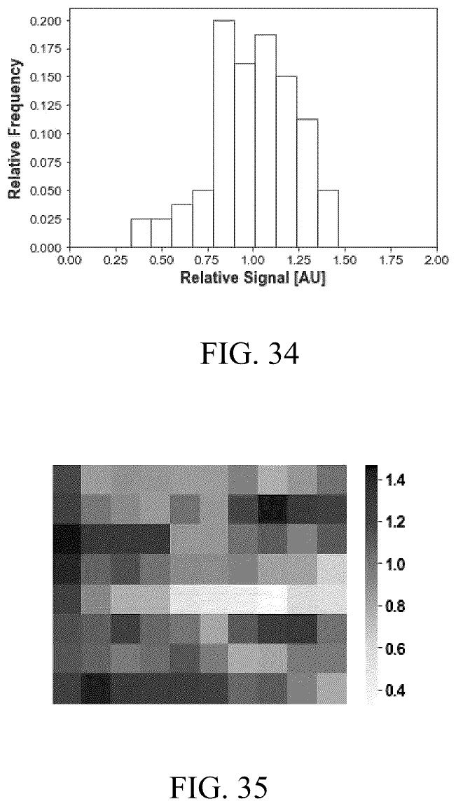

FIG. 34 illustrates the variation in intensities of the bands in FIG. 33 after the bands were subjected to densitometry.

FIG. 35 illustrates the densitometry results after band quantification of the bands in FIG. 33, wherein each band corresponds to a square in the heatmap.

Referring now to FIGS. 36-38, 5 μL of molecular weight ladder 127 was loaded in each well 360 of the first and last columns of the 10% polyacrylamide tris-HCl gel layer 74a, and 4 μL of either recombinant α-Tubulin (˜50 kDa) 128 or HEK293 lysate 129 in the remaining sample wells 360 of the tris-HCl gel layer 74a in 2-fold dilution series. Electrophoresis was conducted, and thereafter, the following procedures were performed as previously described in paragraphs [00207]-[00212] concerning recombinant α-tubulin or HEK293 cell lysate: transfer using PVDF membrane; antibody incubations; and imaged via chemiluminescence. The instant experiment was performed in triplicate.

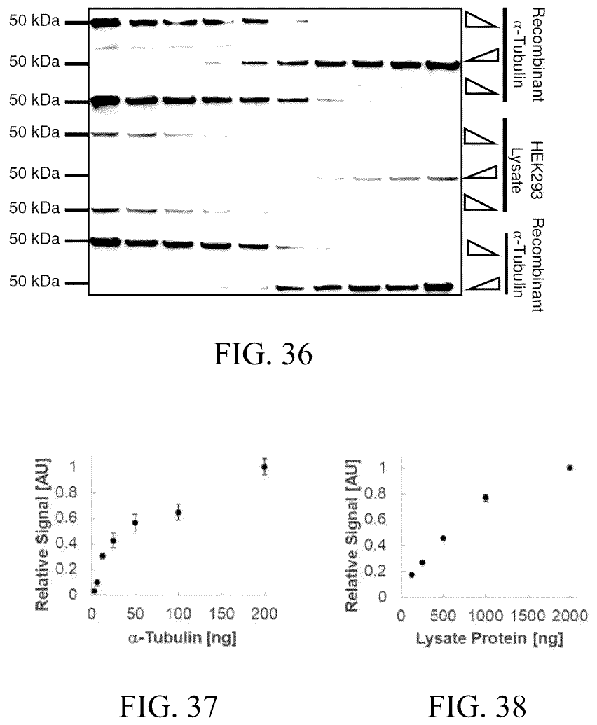

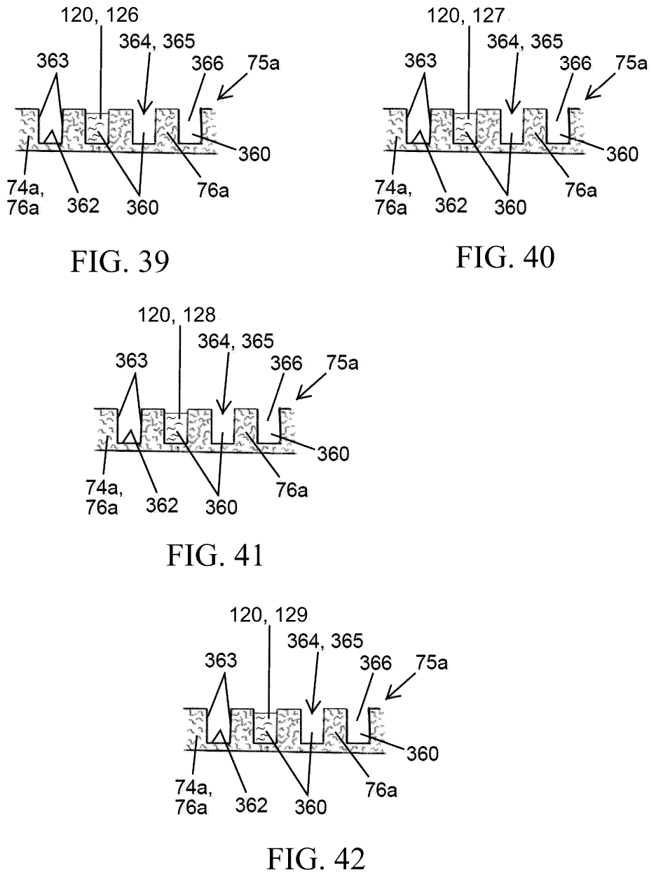

FIG. 36 is a digital image illustrating a full membrane scan resultant corresponding to 5.0 μL of molecular weight ladder samples loaded in each well 360 of the first column and last column (last column not shown) of a 10% Tris-HCl gel layer having 96 sample loading wells 360, and 4 μL of either recombinant α-Tubulin or HEK293 lysate loaded in the remaining sample loading wells 360 in 2-fold dilution series.

FIG. 37 illustrates the variation in intensities of the recombinant α-Tubulin protein bands quantified in FIG. 36 after said bands were subjected to densitometry. The error bars denote standard error from replicates on this blot.

FIG. 38 illustrates the variation in intensities of the HEK293 lysate bands quantified in FIG. 36 after said bands were subjected to densitometry.

It is to be understood that the embodiments and claims are not limited in application to the details of construction and arrangement of the components set forth in the description and/or illustrated in drawings. Rather, the description and/or the drawings provide examples of the embodiments envisioned, but the claims are not limited to any particular embodiment or a preferred embodiment disclosed and/or identified in the specification. Any drawing figures that may be provided are for illustrative purposes only, and merely provide practical examples of the invention disclosed herein. Therefore, any drawing figures provided should not be viewed as restricting the scope of the claims to what is depicted.

The embodiments and claims disclosed herein are further capable of other embodiments and of being practiced and carried out in various ways, including various combinations and sub-combinations of the features described above but that may not have been explicitly disclosed in specific combinations and sub-combinations.

Accordingly, those skilled in the art will appreciate that the conception upon which the embodiments and claims are based may be readily utilized as a basis for the design of other structures, methods, and systems. In addition, it is to be understood that the phraseology and terminology employed herein are for the purposes of description and should not be regarded as limiting the claims.

Furthermore, the purpose of the foregoing Abstract is to enable the U.S. Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way. It is intended that the application is defined by the claims appended hereto.

What is claimed is:

1. A high-throughput multiplexing system for conducting electrophoresis separation of proteins, the system comprising:
a gel casting device comprising a top portion and a bottom portion, the bottom portion providing a gel mold support base, the gel mold support base is seated atop the top portion so as to form an interior gel casting chamber; and
a horizontal electrophoresis tank comprising a polymerized gel layer and gel mold support base support compartment being sizably-shaped, and dimensioned to receive a collaborative structure in a snug, complementary-fit manner, the collaborative structure comprises a sample-loaded polymerized gel layer positioned superjacent a gel mold support base.

2. The system of claim 1, wherein the top portion comprises a sample wells molding portion, the sample wells molding portion comprises a floor having an upper surface opposing a lower surface, the lower surface having a perimeter from which a first longitudinal sidewall, a second longitudinal sidewall, and a latitudinal sidewall extend integrally upward therefrom forming an integrated wall, the integrated wall has an open end opposing the latitudinal sidewall, and wherein the open end includes a base support wall positioned adjacent the open-end edge of the floor and intermediate the first longitudinal sidewall and the second longitudinal sidewall, thereby forming a first portal and a second portal through which an unpolymerized, flowable separation medium is introduced into the gel casting chamber.

3. The system of claim 2, wherein the top portion comprises a plurality of protrusions integrally projecting upwardly from the upper surface of the floor, the plurality of protrusions interpolate the unpolymerized, flowable separation medium.

4. The system of claim 3, wherein the unpolymerized, flowable separation medium comprises a polyacrylamide gel solution.

5. The system of claim 4, wherein the gel casting device is positioned in a vertical orientation such that the first portal and the second portal are facing upwards and providing a loading edge, the casting device is held in such vertical orientation via a vise, and the loading edge having at least one clamp placed and tightened on the loading edge.

6. The system of claim 5, wherein the gel casting device is maintained in the vertical orientation for a period of time enabling polymerization of the polyacrylamide gel solution, thereby producing a polymerized gel layer.

7. The system of claim 6, wherein the polymerized gel layer comprises a plurality of sample loading wells formed integrally therein via the plurality of protrusions.

8. The system of claim 7, wherein the plurality of sample loading wells comprises a number equal to the number of the plurality of protrusions.

9. The system of claim 8, wherein each of the plurality of sample loading wells comprises a sample loading volume measuring in a range comprising approximately 1 μl to 1 ml.

10. The system of claim 3, wherein the top portion comprises a continuous, support shelf integrally recessed from the first longitudinal sidewall, the latitudinal sidewall, and the second longitudinal sidewall for seating the gel mold support base in a complementary-fit manner.

11. The system of claim 1, wherein the horizontal electrophoresis tank comprises a bottom wall having a perimetric interface from which a right sidewall, a left sidewall, a forward sidewall, and a rear sidewall extend upward integrally enclosing the bottom wall and forming a receptacle for containing a buffer solution, and wherein the polymerized gel layer and gel mold support base support compartment is positioned intermediate along the bottom wall of the tank.

12. The system of claim 11, wherein the compartment comprises a first longitudinal sidewall opposing a second longitudinal sidewall, and a first latitudinal sidewall opposing a second latitudinal sidewall, the first longitudinal sidewall and the second longitudinal sidewall each comprises a recessed portion aligned intermediate along the first longitudinal sidewall and the second longitudinal sidewall, respectively, wherein the recessed portions provide spaces enabling the collaborative structure to be grasped by a user in order to be seated in and removed from the compartment.

13. The system of claim 9, wherein each of the plurality of sample loading wells is loaded sequentially with a liquid sample comprising a volume measuring in a range comprising approximately 0.1 μl to 1 ml of a liquid sample.

14. The system of claim 12, wherein the collaborative structure is seated within the compartment of the horizontal electrophoresis tank, the receptacle of the horizontal electrophoresis tank is filled with the buffer solution to a depth so as to completely submerge the polymerized gel layer of the collaborative structure.

15. The system of claim 14, wherein the buffer solution having an electric field applied thereto causing an electric current to pass through the buffer solution, the liquid samples, and through the polymerized gel layer so as to facilitate separation of proteins of the liquid samples.

16. The system of claim 7, wherein the polymerized gel layer comprises a thickness measuring in a range comprising approximately 0.5 mm to 20 mm.

17. The system of claim 9, wherein each of the plurality of sample loading wells is loaded sequentially with a liquid sample, the liquid sample comprising a volume measuring in a range comprising approximately 0.1 μL to 1 mL of molecular weight ladder being loaded in each sample loading well of a first column and a last column of the polymerized gel layer, and a lysate being loaded in each sample loading well positioned between the first column and the last column of the polymerized gel layer.

18. A method for conducting horizontal electrophoresis separation of molecules, the method comprising the steps of:
removing a collaborative structure from a gel casting device;
loading sequentially each of a plurality of sample loading wells of a polymerized gel layer of the collaborative structure with a volume of a liquid sample via at least one of a manual micropipette instrument and an automated microliter multi-pipette sample loading mechanism, thereby providing a sample-loaded polymerized gel layer;
seating a collaborative structure in a polymerized gel layer and gel mold support base support compartment of a horizontal electrophoresis tank;
filling a receptacle of the horizontal electrophoresis tank with a buffer solution to a depth so as to completely submerge the polymerized gel layer;
applying an electric field at approximately 50 volts to the buffer solution so that an electric current passes through the buffer solution, and through the sample-loaded polymerized gel layer for a period of time;
transferring the collaborative structure into a plastic tray containing a cold transfer buffer;
incubating the collaborative structure over a period of time;
transferring the sample-loaded polymerized gel layer of the collaborative structure to a transfer sandwich cassette;
placing the transfer sandwich cassette into a blotter tank having a low temperature measure;
filling the blotter tank with a cold transfer buffer;
stirring the blotter tank magnetically; and
subjecting the transfer sandwich cassette in the blotter tank to a constant electric field measure for a period of time at room temperature; and
removing a protein-staining membrane from the transfer sandwich cassette.

19. The method of claim 18, further comprising the steps of:
placing the protein-staining membrane onto a chemi tray;
scanning the protein-staining membrane via a digital scanning and imaging device; and
producing digital images of chemiluminescence of the molecular weight ladder bands.

20. The method of claim 18, further comprising the steps of:
placing the protein-staining membrane immediately into a glass dish containing a volume of a room temperature blocking buffer;
subjecting the protein-staining membrane, while positioned in the glass dish, to agitation for a period of time;
incubating the protein-staining membrane with a volume of a primary antibody solution at room temperature while concurrently subjecting the protein-staining membrane to agitation for a period of time;
washing the protein-staining membrane multiple times with a volume of Tris buffered saline with Tween-20 for a period of time and agitated at room temperature for a period of time;
incubating the protein-staining membrane with a volume of a secondary antibody solution for a period of time and agitated for a period of time at room temperature;
washing the protein-staining membrane multiple times with a volume of TBS-T for a period of time and agitated for a period of time at room temperature;
applying a volume of a chemiluminescent substrate solution dropwise to a top of the protein-staining membrane;
placing the protein-staining membrane onto a chemi tray;
scanning the protein-staining membrane via a digital scanning and imaging device; and
producing digital images of chemiluminescence of the molecular weight ladder bands.
